SEQUENCE LISTING (14 sequences)

SEQ ID NO: 1 - DNA, 18 bp, Artificial Sequence
  Synthetic Construct
agcattcccttgacacag

SEQ ID NO: 2 - DNA, 18 bp, Artificial Sequence
  Synthetic Construct
ggtctacagtgatgttgc

SEQ ID NO: 3 - DNA, 21 bp, Artificial Sequence
  Synthetic Construct
tgcagctgccactcagccggg

SEQ ID NO: 4 - DNA, 23 bp, Artificial Sequence
  Synthetic Construct
tgccaagccagtccattctccac

SEQ ID NO: 5 - DNA, 20 bp, Artificial Sequence
  Synthetic Construct
gaacctgctcttctctgagt

SEQ ID NO: 6 - DNA, 18 bp, Artificial Sequence
  Synthetic Construct
agctgaggaggtacagtg

SEQ ID NO: 7 - DNA, 19 bp, Artificial Sequence
  Synthetic Construct
tgctgtcactgatgctgct

SEQ ID NO: 8 - DNA, 20 bp, Artificial Sequence
  Synthetic Construct
ggcttcgtctacagcgtctt

SEQ ID NO: 9 - DNA, 20 bp, Artificial Sequence
  Synthetic Construct
tacaggaagctgctggaagg

SEQ ID NO: 10 - DNA, 19 bp, Artificial Sequence
  Synthetic Construct
tgggtgtcaaccagaggaa

SEQ ID NO: 11 - DNA, 22 bp, Artificial Sequence
  Synthetic Construct
caaagccacaatgttccaccag

SEQ ID NO: 12 - DNA, 22 bp, Artificial Sequence
  Synthetic Construct
gatgtcgtcgtcagacctctcg

SEQ ID NO: 13 - DNA, 20 bp, Artificial Sequence
  Synthetic Construct
cccactaacatcaaatgggg

SEQ ID NO: 14 - DNA, 20 bp, Artificial Sequence
  Synthetic Construct
gatgatgacccttttggctc

(19) United States
(12) Patent Application Publication
(10) Pub. No.: US 2022/0096526 A1
(43) Pub. Date: Mar. 31, 2022

(54) METHODS AND COMPOSITIONS FOR THE TREATMENT AND PREVENTION OF OCULAR DISEASES AND CONDITIONS

(71) Applicant: Tufts Medical Center, Inc., Boston, MA (US)

(72) Inventors: Pedram HAMRAH, Wellesley, MA (US); Arsia JAMALI, Boston, MA (US)

(21) Appl. No.: 17/425,058

(22) PCT Filed: Jan. 22, 2020
(86) PCT No.: PCT/US2020/014573

§ 371 Date: Jul. 22, 2021

Related U.S. Application Data
(60) Provisional application No. 62/795,157, filed on Jan. 22, 2019.

(51) Int. Cl. A61K 31/7125; A61P 27/02; A61K 9/00

(52) U.S. Cl. CPC A61K 31/7125; A61K 9/0048; A61P 27/02

(57) ABSTRACT

The invention relates to methods and compositions for use in the treatment and prevention of ocular diseases and conditions.

### STATEMENT AS TO FEDERALLY FUNDED RESEARCH

This invention was made with government support under NIH R21-EY025939-01 and NIH R01-EY022695 awarded by National Institutes of Health. The government has certain rights in the invention.

### FIELD OF THE INVENTION

The invention relates to methods and compositions for use in the treatment and prevention of ocular diseases and conditions.

### BACKGROUND OF THE INVENTION

Plasmacytoid dendritic cells (pDCs) are a unique subpopulation of bone marrow derived cells that are morphologically and functionally distinct from conventional (or classical) dendritic cells (cDCs) (McKenna et al., J. Virol. 79(1):17-27, 2005; Reizis et al., Annu. Rev. Immunol. 29:163-183, 2011). In contrast to cDCs, which are considered as professional antigen presenting cells, pDCs exhibit less capacity for antigen presentation under steady state (McKenna et al., J. Virol. 79(1):17-27, 2005; Reizis et al., Annu. Rev. Immunol. 29:163-183, 2011). pDCs are further distinguished from cDCs by expression of a distinct set of markers including CD11c^{low}, PDCA-1, B220, and Siglec-H in mice (Colonna et al., Nat. Immunol. 5(12):1219-1226, 2004; Blasius et al., Blood 107(6):2474-2476, 2006). Nevertheless, similar to their conventional counterparts, pDCs bridge innate and adaptive immune responses in versatile settings by secreting a variety of cytokines including TNF-α, IL-8, and IL-12 (McKenna et al., J. Virol. 79(1):17-27, 2005; Colonna et al., Nat. Immunol. 5(12):1219-1226, 2004; Ito et al., J. Exp. Med. 195(11):1507-1512, 2002).

pDCs express a characteristic repertoire of endosomal receptors, including Toll-like receptor 7 (TLR7) and TLR9. These receptors enable pDCs to detect single-stranded RNA and double-stranded unmethylated CpG-containing DNA, respectively (McKenna et al., J. Virol. 79(1):17-27, 2005; Swiecki et al., Nat. Rev. Immunol. 15(8):471-485, 2015). Although pDCs were originally accredited for their powerful interferon-alpha (IFN-α) secretion capacity, further studies revealed diverse functions of pDCs (Reizis et al., Annu. Rev. Immunol. 29:163-183, 2011; Swiecki et al., Nat. Rev. Immunol. 15(8):471-485, 2015). Upon activation, pDCs up-regulate expression of MHC-II and co-stimulatory molecules, including CD40, CD80, and CD80, and gain capacity to capture, process, and present antigens to T cells (McKenna et al., J. Virol. 79(1):17-27, 2005; Grouard et al., J. Exp. Med. 185(6):1101-1111, 1997; Mouries et al., Blood 112(9):3713-3722, 2008; Fonteneau et al., Blood 101(9):3520-3526, 2003; Schlecht et al., Blood 104(6):1808-1815,2004; Cella et al., Nat. Immunol. 1(4):305-310, 2000; Young et al., Nat. Immunol. 9(11):1244-1252, 2008). These cells can promote recruitment of immune cells to the site of inflammation by secreting CXC-chemokine ligand 8 (CXCL8; IL-8), CXCL10, CC-chemokine ligand 3 (CCL3), CCL4, and CCL5 (Swiecki et al., Nat. Rev. Immunol. 15(8):471-485, 2015; Piqueras et al., Blood 107(7):2613-2618, 2006; Decalf et al., J. Exp. Med. 204(10):2423-2437, 2007). Also, they promote NK cell activation by IL-12 and IL-18, and drive plasma cell antibody production through IFN-α and IL-6 (Swiecki et al., Nat. Rev. Immunol. 15(8):471-485, 2015; Jego et al., Immunity 19(2):225-234, 2003; Poeck et al., Blood 103(8):3058-3064, 2004). pDCs are also implicated in induction of tolerance to dietary antigens in the gut and to grafts by multiple mechanisms including expression of 2,3-dioxygenase, promotion of CD4+ and CD8+ regulatory T cells, eliminating antigen-specific CD8+ T cells, and suppressing CD4+ and CD8+ T cell-mediated delayed-type hypersensitivity reactions (Munn et al., J. Immunol. 172(7):4100-4110, 2004; Dubois et al., Gastroenterology 137(3):1019-1028, 2009; Goubier et al., Immunity 29(3):464-475, 2008; Li et al., J. Immunol. 185(2):823-833, 2010; Manlapat et al., Eur. J. Immunol. 37(4):1064-1072, 2007; Martin-Gayo et al., Blood 115(26):5366-5375, 2010; Moseman et al., J. Immunol. 173(7):4433-4442, 2004).

Communication between the immune and peripheral nervous systems has been appreciated for several decades (Payan et al., Adv. Immunol. 39:299-323, 1986; Petrov et al., Ann. N. Y. Acad. Sci. 496:271-277, 1987; Payan et al., Ann. N. Y. Acad. Sci. 496:182-191, 1987). In this regard, anatomical connections of nerves and immune cells have been widely described in various peripheral tissues. For instance, mast cells, eosinophils, and plasma cells, as cellular components of both innate and adaptive immune responses, are observed in close association with neural processes in normal gut and heart (Arizono et al., Lab. Invest. 62(5):626-634, 1992; Stead, Reg. Immunol. 4(2):91-99, 1992). Similarly, apparent contacts are documented between 70-80% of CD1+ Langerhans cells and epidermal CGRP+ axons in skin (Hosoi et al., Nature 363(6425):159-163, 1993). Also, macrophages are observed among endoneurial cells of peripheral neurons (Oldfors et al., Acta Neuropathol. 49(1):43-49, 1980; Monaco et al., J. Neurocytol. 21(9):623-634, 1992; Mueller et al., Am. J. Pathol. 159(6):2187-2197, 2001). Nevertheless, despite our well-established knowledge on propinquity of immune cells and nerves, unravelling the cellular and molecular interactions between these systems is crucial to facilitate neuronal regeneration and to target destructive inflammatory responses.

Cornea is the most densely innervated tissue in the body, with a sensitivity of approximately 300-600 times more than the skin (Al-Aqaba et al., Br. J. Ophthalmol. 94(6):784-789, 2010; Marfut et al., Exp. Eye Res. 90(4):478-492, 2010; Marfurt et al., J. Comp. Neurol. 336(4):517-531, 1993; Mullet et al., Invest. Ophthalmol. Vis. Sci. 37(4):476-488, 1996; Muller et al., Invest. Ophthalmol. Vis. Sci. 38(5):985-995, 1997; Millodot et al., Ophthalmic. Physiol. Opt. 4(4):305-318, 1984). Corneal sensory nerves, originating from the ophthalmic branch of trigeminal nerve, can be classified into two groups: (1) sub-basal nerve plexus, which is the most densely innervated region of the cornea and runs parallel to the superficial corneal surface, between Bowman's layer and the basal epithelium, and (2) stromal plexus, which consists of thicker nerve fibers in the corneal stroma. Notably, corneal innervation is currently under more thoughtful investigation as recent studies have provided clues for participation of these structures in pathophysiology of various ocular surface diseases including dry eye and neurotropic keratitis (Bonini et al., Eye (London) 17(8):989-995, 2003; Dastjerdi et al., Int. Ophthalmol. Clin. 49(1):11-20, 2009). Despite the immune privilege of cornea, this transparent tissue hosts different subtypes of immune cells in steady state, including Langerhans cells, cDCs, macrophages, and recently identified pDCs (Hamrah et al., J. Leukoc. Biol. 74(2):172-178, 2003; Hamrah et al., Chem. Immunol. Allergy 92:58-70, 2007). Dense innervation of the cornea, feasibility of clinical assessment of sensory nerve functions, and presence of resident immune cells in close contact with neural network in the cornea make this tissue a unique environment to study neuroimmune interactions.

### SUMMARY OF THE INVENTION

The invention provides methods of preventing or treating a disease or condition of the eye in a subject (e.g., a human subject). The methods include administering a toll-like receptor 7 (TLR7) agonist to an eye of the subject. In various examples, the disease or condition of the eye is characterized by neovascularization (e.g., corneal, retinal, or choroidal neovascularization). In various examples, the subject has or is at risk of developing corneal infection, inflammation, autoimmune disease, limbal stem cell deficiency, neoplasm, dry eye disease, or ocular damage due to trauma, surgery, or contact lens wear.

The invention also includes methods of preventing or treating a disease or condition of the eye in a subject (e.g., a human subject), which include administering a toll-like receptor 9 (TLR9) agonist to an eye of the subject, wherein the subject has or is at risk of developing corneal infection, inflammation, autoimmune disease, limbal stem cell deficiency, neoplasm, dry eye disease, or ocular damage due to trauma, surgery, or contact lens wear; or the disease or condition is characterized by retinal or choroidal neovascularization.

In various examples of the methods of the invention, the subject has or is at risk of developing ischemic retinopathy, diabetic retinopathy, retinopathy of prematurity, retinal vein occlusion, or macular degeneration. In various examples, the subject has or is at risk of developing inflammatory neovascularization with uveitis. In various examples, the disease or condition of the eye is characterized by ocular nerve degeneration or damage (e.g., corneal nerve damage). In various examples, the subject has or is at risk of developing dry eye disease, corneal infection, or corneal neurotrophic keratopathy. In various examples, the subject has or is at risk of experiencing ocular damage due to trauma, surgery, or contact lens wear.

Optionally, a TLR7 agonist used in the methods and compositions of the invention is selected from the group consisting of imiquimod, resiquimod, loxoribine, gardiquimod, and vesatolimod (GS-9620), GS-986, and combinations of two or more thereof.

Optionally, a TLR9 agonist used in the methods and compositions of the invention is a CpG oligonucleotide [CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10] or a combination thereof.

The methods of the invention can further include administering a plasmacytoid dendritic cell (pDC) to an eye of the subject (e.g., a human subject). In various embodiments, the plasmacytoid dendritic cell is applied to the cornea of the subject and/or is administered to the subject by intravitreal or sub-retinal injection. In various examples, the plasmacytoid dendritic cell is obtained from the subject to whom it is later administered. In other embodiments, the plasmacytoid dendritic cell is obtained from an individual and/or species different from the subject to whom it is administered.

Optionally, the methods of the invention can include the administration of a TLR7 agonist in combination with a TLR9 agonist. Examples of these compounds are provided above an elsewhere herein.

The invention further includes compositions including one or more TLR7 agonist and/or a TLR9 agonist and an ophthalmic carrier or diluent. Optionally, the carrier or diluent is selected from the group consisting of glycerin, hydroxyethylcellulose (HEC), hydroxypropyl methylcellulose (HPMC), polyvinyl alcohol (PVA), carboxy methylcellulose (CMC), sodium chloride, polyvidone, polyethylene glycol, propylene glycol, hypromelloses, boric acid, sodium borate, sodium hyaluronate, Hamamelis virginiana, and tissue glue. In some embodiments, the compositions further include a preservative, which is optionally selected from the group consisting of benzalkonium (BAK), poloxamer 407, potassium sorbate, polyquad, sodium perborate, purite, cetrimide, hydroxypropyl guar, and polyquaternium.

The invention also provides kits that include one or more composition, as described herein, optionally in combination with a topical anesthetic eye drop, and/or a syringe, dropper, or applicator for administration of the composition.

The invention additionally provides compositions and kits for use in methods such as those described herein, as well as use of the compositions and kits in the preparation of medicaments for carrying out such methods.

The invention provides several advantages. For example, the methods provide improved approaches to preventing diseases and conditions associated with ocular neovascularization and nerve damage, as described herein. In addition, as the methods of the invention can involve the use of local administration, lower amounts of total drug can be used than if the drugs were to be administered systemically. This leads to reduced incidence of any possible side effects of the drugs, as well as decreased costs.

Other features and advantages of the invention will be apparent from the following detailed description, the drawings, and the claims.

### BRIEF DESCRIPTION OF THE DRAWINGS

FIGS. 1A-1G. Depletion of plasmacytoid dendritic cells is accompanied by abrupt corneal nerve degeneration and sensory function diminishment. (FIG. 1A) Confocal micrograph of naïve WT C56BL/6 corneal whole mount demonstrates spatial proximity of resident pDCs, identified by expression of CD45 (red; second panel) and PDCA-1 (green; first panel), and corneal nerves (white). Scale bar, 100 μm. (FIG. 1B) Confocal micrograph of corneal whole mount stained with PDCA-1 (pDC marker) in BDCA2-DTR mouse in steady state, 36 hours, and 3 days following subconjunctival injection of 30 ng DT, showing depletion of pDCs in the BDCA2-DTR cornea after exposure to DT. (FIG. 1C) Flow cytomeric histogram of digested corneas of DT-treated WT C57BL/6 (blue) and BDCA2-DTR mice (pDC-depleted; red) as well as PBS-administered BDCA2-DTR mice (green). Numbers represent the frequency of PDCA-1⁺CD11c^{low}CD11b^{neg}, CD11b⁺, F4/80⁺, CD11c⁺ populations in total CD45⁺ cells. n=6-8 in each group. (FIGS. 1D-1F) Local depletion of corneal pDCs by subconjunctival injection of DT in BDCA2-DTR mice is accompanied by degeneration of sub-basal and stromal nerve plexuses of central (FIGS. 1D and 1E) and peripheral cornea (FIG. 1F). Nerve plexuses in control groups, consisting of WT C57BL/6 mice receiving DT and BDCA2-DTR mice treated with PBS, remained intact (FIGS. 1D-1F). Confocal micrograph of the center of whole-mounted corneas stained with (βIII-Tubulin (a pan-neuronal marker); (FIGS. 1E and 1F) Quantification of the corneal nerve density. Scale bar in (FIG. 1D), 100 μm. Error bars show SD, n=3-4 in each group. *, p<0.05; ***, p<0.001. P values are calculated by ANOVA with Bonferroni post hoc. (FIG. 1G) Frequency of intact corneal blink reflex is diminished in the central cornea following pDC depletion versus control groups. Bars show SD of 3 independent experiments, n=3-5 in each group in each experiment. * p<0.01; ** p<0.001. P values are calculated by Chi square.

FIGS. 2A-2D. Repopulation of plasmacytoid dendritic cells after initial depletion induces nerve regeneration in cornea and re-establishes corneal sensory function. (FIG. 2A) Confocal micrograph of whole-mounted corneas stained with βIII-Tubulin (a pan-neuronal marker) in center (upper panel) and periphery (lower panel), 5 and 14 days following stopping DT injection (pDC repopulation). Scale bar, 100 μm. (FIGS. 2B and 2C) Quantification of corneal nerve density 5 and 14 following stopping DT injections (pDC repopulation) in center (FIG. 2B) and periphery (FIG. 2C) of cornea. Error bars show SD, n=3-4 in each group. *, p<0.001. P values are calculated by ANOVA with Bonferroni post hoc. (FIG. 2D) Frequency of intact corneal blink reflex in the central cornea following pDC repopulation (left: +5d pDC repopulation; right: +14d pDC repopulation). Bars show SD of 3 independent experiments, n=3-5 in each group in each experiment. * p<0.001. P values are calculated by Chi square.

FIGS. 3A-3F. Plasmacytoid dendritic cells are a vital source of NGF in cornea. (FIG. 3A) Relative NGF mRNA levels in corneal stroma in naïve, pDC depleted, and control DT administered WT C57BL/6 mice, as well as upon re-population of pDCs. Bars show SD of 3 independent biological experiments, each on pooled 6-8 corneal stoma per group. P values are calculated by ANOVA with Bonferroni post hoc. (FIG. 3B) Representative FACS analysis of sorted GFP-tagged pDCs from solenocytes of transgenic DPE-GFP×RAG1^{-/-} mice stained for NGF. (FIG. 3C) Agarose gel electrophoresis on PCR product with NGF primer on the cDNA synthetized from the RNA extracted from sorted splenic pDCs from naïve DPE-GFP×RAG1^{-/-} mouse (3 different samples) or control lacking template RNA. Image is representative of 3 biologic repeats. (FIG. 3D) Representative confocal micrograph of whole-mount WT C57BL/6 naïve cornea stained with CD45 (green; pan-leukocyte marker), PDCA-1 (white; pDC marker), and NGF (red) highlights co-staining of pDCs and NGF in the cornea. Scale bar, 50 μm. (FIG. 3E) Representative FACS analysis of naïve, 3 day post thermal cautery, and 7 day sutured single corneal cells, following removing debris, dead cells, and doublets, and gating on CD45 and PDCA-1 shows co-localization of pDCs (CD45⁺PDCA-1⁺D45R/B220⁺) with NGF. (FIG. 3F) Relative NGF mRNA level in purified splenic cDCs and pDCs. Bars show SD of 3 independent biological experiments. * p<0.01. P value is calculated with T test.

FIGS. 4A-4D. Plasmacytoid dendritic cells promote neurite outgrowth in trigeminal ganglion cell culture through secretion of nerve growth factor in vitro. (FIG. 4A) Imaged TGCs stained with 1 μM calcein following 3 days co-culture without and with indicated number of pDCs. Scale bar, 50 μm. (FIG. 4B) Quantified neurite outgrowth per soma of TGCs following 3 days co-culture without and with indicated number of pDCs. Bars show SD of 3 independent experiments, each in triplicate. * p<0.001. P values are calculated by ANOVA with Bonferroni post hoc. (FIG. 4C) Relative mRNA levels of Sprr1a, GAP43, Vimentin, and BDNF in TGCs following 3 days co-culture without and with indicated number of pDCs. Each set of bars corresponds, in order, to the pDC cell amounts indicated at the top of the graph. Bars show SD of 3 independent experiments, each in triplicate. ‡ p<0.05; * p<0.001. P values are calculated by ANOVA with Bonferroni post hoc. (FIG. 4D) NGF protein level in the cell culture media following 3 days co-culture of TGCs and indicated number of pDCs as well as in TGCs or pDCs mono-culture. Bars show SD of 3 independent experiments. ‡ less than detection limit. * p<0.001. P values are calculated by ANOVA with Bonferroni post hoc.

FIGS. 5A and 5B. Plasmacytoid dendritic cell-induced neurite outgrowth of trigeminal ganglion cells is partially dependent on NGF. (FIG. 5A) Micrographs of TGCs stained with 1 μM calcein following 5 days co-culture with 5000 pDCs or without pDCs (control) with indicated concentration of anti-NGF blocking antibody. Scale bar, 50 μm. (FIG. 5B) Quantification of neurite outgrowth per soma of TGCs following 5 days co-culture with 5000 pDCs or without pDCs (controls) with indicated concentration of anti-NGF blocking antibody. Bars show SD of 3 independent experiments, each in triplicate. * p<0.001. P values are calculated by ANOVA with Bonferroni post hoc.

FIGS. 6A-6E. Stimulation of plasmacytoid dendritic cells via toll-like receptor 7 enhances production of NGF and subsequently promotes neurite outgrowth. (FIG. 6A) NGF relative mRNA levels in sorted splenic pDCs following 3 days stimulation with indicated density of Imiquimod (TLR7 agonist) in vitro. Bars show SD of 3 independent experiments, each in triplicate. * p<0.001. P values are calculated by ANOVA with Bonferroni post hoc. Bars are in the same order as indicated at the top of the graph. (FIGS. 6B and 6C) NGF relative mRNA levels in sorted corneal pDCs 24 hours following stimulation with 3 μg/ml Imiquimod, 3 μg/ml CpG ODN, or control ODN in vitro (FIG. 6B) as well as 24 hours following inoculation of 10 μg Imiquimod, or 10 μg CpG ODN, or control ODN in vivo (FIG. 6C). Bars show SD of 3 biological repeats in triplicate. * p<0.001. P values are calculated by ANOVA with Bonferroni post hoc. Bars are in the same order as indicated at the top of the graph. (FIG. 6D) Micrographs of TGCs stained with 1 μM calcein following 3 days co-culture with 8000 pDCs or TGCs mono-culture (controls) with indicated concentration of Imiquimod. Scale bar, 50 μm. (FIG. 6E) Quantification of neurite outgrowth per soma of TGCs following 3 days co-culture with 8000 pDCs or TGCs mono-culture with indicated concentration of Imiquimod. Bars show SD of 3 independent experiments, each in triplicate. * p<0.001. P values are calculated by ANOVA with Bonferroni post hoc.

FIGS. 7A-7E. Enhanced production of NGF following stimulation of plasmacytoid dendritic cells via TLR7 is mediated through phosphorylation of CREB by p38 MAP kinase. (FIG. 7A) CREB relative mRNA levels in sorted splenic pDCs following 3 days stimulation with indicated density of Imiquimod (TLR7 agonist) in vitro. Bars show SD of 3 independent experiments, each in triplicate. * p<0.001. P values are calculated by ANOVA with Bonferroni post hoc. Bars are in the same order as indicated at the top of the graph. (FIGS. 7B and 7C) CREB relative mRNA levels in sorted corneal pDCs 24 hours following stimulation with 3 μg/ml Imiquimod, 3 μg/mICpG ODN, or control ODN in vitro (FIG. 7B) as well as 24 hours following inoculation of 10 μg Imiquimod or control in vivo (FIG. 7C). Bars show SD of 3 biological repeats in triplicate. * p<0.001. P values are calculated by ANOVA with Bonferroni post hoc. Bars are in the same order as indicated at the top of the graph. (FIG. 7D) Relative protein level of phospho-CREB normalized to β-actin in sorted splenic pDCs 3 days after culture. Cells were pre-treated for 1 hour with either 10 μM SB203580 (p38 MAP kinase inhibitor) or PBS and were subsequently treated with 1 μg Imiquimod or PBS. Bars show SD of 4 independent experiments. * p<0.001. P values are calculated by ANOVA with Bonferroni post hoc. (FIG. 7E) Relative mRNA levels of NGF and CREB in sorted splenic pDCs 3d after culture. Cells were pre-treated for 1 hour with either 10 μM SB203580 (p38 MAP kinase inhibitor) or PBS and were subsequently treated with 1 μg Imiquimod or PBS. Bars show SD of 3 independent experiments, each in triplicate. * p<0.001. P values are calculated by ANOVA with Bonferroni post hoc. Bars are in the same order as indicated at the top of the graph.

FIGS. 8A-8E. Treatment of splenic cells with TLR7 or TLR9 agonists enhances expression of endostatin in pDCs. (FIGS. 8A-8D) Gating strategy for selecting plasmacytoid dendritic cells among isolated splenic cells. Flow cytometry dot plots demonstrating dead cells, debris (FIG. 8A) and doublets (FIG. 8B) were excluded using forward and side scatters to only include live single cells. CD45⁺ immune cells were then selected (FIG. 8C) and pDCs were identified by expression of PDCA-1 and CD45R/B220 (FIG. 8D). (FIG. 8E) Flow cytometry histogram shows that the frequency of endostatin-secreting pDCs increases following 24 hours of treatment with TLR7 or TLR9 agonist. Further, median fluorescent intensity of endostatin staining is enhanced following stimulating pDCs with aforementioned agonists, showing augmentation of endostatin levels in pDCs. Numbers present the percentage (median fluorescent intensity) of endostatin expression in pDCs.

### DETAILED DESCRIPTION

The invention provides methods and compositions for use in preventing or treating diseases and conditions of the eye by administration of one or more TLR7 and/or TLR9 agonist to the eye. The methods and compositions of the invention can be used to prevent or treat diseases or conditions characterized by, e.g., neovascularization of one or more tissues of the eye including, e.g., the cornea, the retina, or the choroid. In addition, with respect to TLR7 agonists, in particular, the methods and compositions can also be used to prevent or treat diseases or conditions characterized by ocular (e.g., corneal) nerve degeneration or damage. Central to the invention are the discoveries that TLR7 and TLR9 agonists can be used to reduce or limit neovascularization in the eye, and also that TLR7 agonists can be used reduce or limit corneal nerve damage and promote corneal nerve regeneration. The methods and compositions of the invention are described further, as follows.

### Identification of Subjects

Subjects that can be treated using the methods and compositions of the invention include those suffering from, or at risk for, ocular neovascularization and/or ocular nerve degeneration or damage. The subjects include human patients (adults and children) who have or are at risk of developing a disease or condition of the eye as described herein.

Neovascularization is a common feature of many conditions, and may occur in tissues of the eye including, for example, the cornea, retina, or choroid. This process involves new blood vessel formation in abnormal locations, such as the cornea, a normally avascular tissue. Diseases that are characterized by corneal neovascularization include, for example, corneal infection, inflammation, autoimmune disease, limbal stem cell deficiency, neoplasia, dry eye disease, radiation, blepharitis, uveitis, keratitis, corneal ulcers, glaucoma, rosacea, and lupus.

Diseases or conditions that are characterized by retinal neovascularization include, for example, ischemic retinopathies, diabetic retinopathy, retinopathy of prematurity, retinal vein occlusions, ocular ischemic syndrome, sickle cell disease, radiation, and Eales' disease.

Further, diseases or conditions that are characterized by choroidal neovascularization include, for example, inflammatory neovascularization with uveitis, macular degeneration, ocular trauma, trauma due to excessive or improper contact lens wear, sickle cell disease, pseudoxanthoma elasticum, angioid streaks, optic disc drusen, extreme myopia, malignant myopic degeneration, and histoplasmosis.

In addition to the above, trauma, such as surgery, injury, burn (e.g., chemical burn), and excessive or improper contact lens use, can also be characterized by neovascularization. Inflammation associated with ocular (e.g., corneal) neovascularization can result from bacterial and viral infection, Stevens-Johnson syndrome, graft rejection, ocular cicatricial pemphigoid, and degenerative disorders, such as pterygium and Terrien marginal degeneration.

Subjects having or at risk of developing any of the aforementioned disorders or conditions can be treated using the methods and compositions of the invention, focusing on the administration of one or more TLR7 and/or TLR9 agonist.

As noted above, the cornea is the most densely innervated structure in the human body, and is therefore highly sensitive to touch, temperature, and chemical stimulation, all of which are sensed by corneal nerves. Corneal nerves are also involved in blinking, wound healing, tear production, and tear secretion. Damage to or loss of corneal nerves can lead to dry eyes, impairment of sensation, corneal edema, impairment of corneal epithelium healing, corneal ulcerations and erosions, and a cloudy corneal epithelium, among other conditions. Diseases or conditions characterized by corneal nerve degeneration or damage include, for example, dry eye disease, neurotrophic keratitis, corneal infections, excessive or improper contact lens wear, ocular herpes (HSV), herpes zoster (shingles), chemical and physical burns, injury, trauma, surgery (including corneal transplantation, laser assisted in-situ keratomileusis (LASIK), penetrating keratoplasty (PK), automated lamellar keratoplasty (ALK), photorefractive keratectomy (PRK), radial keratotomy (RK), cataract surgery, and corneal incisions), abuse of topical anesthetics, topical drug toxicity, corneal dystrophies, vitamin A deficiency, diabetes, microbial keratitis, and herpetic keratitis. The methods and compositions of the invention can be used to prevent or treat any of the aforementioned diseases or conditions of the eye, focusing on the use of one or more TLR7 agonist.

### TLR7 and TLR9 Agonists

TLR7 agonists that can be used in the invention include, e.g., imiquimod, resiquimod, gardiquimod, loxoribine, vesatolimod (GS-9620), and GS-986. As is understood in the art, some TLR7 agonists (e.g., gardiquimod, imiquimod, and resiquimod) are also TLR8 agonists. Use of such dual agonists (e.g., TLR7/8 agonists) are also included within the scope of the invention.

TLR9 agonists that can be used in the invention include CpG oligonucleotides. In specific non-limiting examples, the following agents can be used: CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

The agonists can be administered in amounts determined to be appropriate by those of skill in the art. Exemplary amounts of TLR7 (or TLR7/8) agonists for administration are one or more drops (e.g., 1, 2, 3, 4, or 5 drops) of a 0.05-10% w/v (e.g., 0.1-8%, 1-6%, 2-5%, or 3-4% w/v) solution, while exemplary amounts of TLR9 agonists are 0.5-100 mg (e.g., 1-75, 3-50, 5-40, 10-30, or 15-25 mg) per dose. Optionally, the TLR7 (including TLR7/8 agonists) and TLR9 agonists are comprised within pharmaceutically acceptable compositions, such as ophthalmic compositions, as known in the art. Examples of such compositions are described below. The agonists are included within these compositions in amounts sufficient to provide a desired dosage, using a desired volume (e.g., the volume of a drop from a standard eye dropper), as can be determined by those of skill in the art.

### Compositions

Compositions of the invention include the agents described herein (e.g., TLR7 and/or TLR9 agonists; also dual agonists; see, e.g., above) in an ophthalmic administrable form. The compositions can thus include the agent in the form of, e.g., an aqueous solution, a gel, or a cream, which may include, e.g., one or more of the following excipients: glycerin, hydroxyethylcellulose (HEC), hydroxypropyl methylcellulose (HPMC), polyvinyl alcohol (PVA), carboxy methylcellulose (CMC), sodium chloride, polyvidone, polyethylene glycol, propylene glycol, hypromelloses, boric acid, sodium borate, sodium hyaluronate, and Hamamelis virginiana, optionally in combination with one or more preservative (e.g., benzalkonium (BAK), poloxamer 407, potassium sorbate, polyquad, sodium perborate, purite, cetrimide, hydroxypropyl guar, or polyquaternium).

In various specific examples, the compositions may include glycerin (0.1-3% v/v, e.g., 0.1%, 0.2%, 0.25%, 0.3%, 0.4%, 0.5%, 0.75%, 1.0%, 1.5%, 2.0%, 2.5%, or 3.0% v/v), optionally in combination with propylene glycol (0.1-3% v/v, e.g., 0.1%, 0.2%, 0.25%, 0.3%, 0.4%, 0.5%, 0.75%, 1.0%, 1.5%, 2.0%, 2.5%, or 3% v/v), polyethylene glycol (e.g., PEG400; 0.1-3% v/v, e.g., 0.1%, 0.2%, 0.25%, 0.3%, 0.4%, 0.5%, 0.75%, 1.0%, 1.5%, 2.0%, 2.5%, or 3.0% v/v), and/or hypromelloses (0.1-3% w/v, e.g., 0.1%, 0.2%, 0.25%, 0.3%, 0.4%, 0.5%, 0.75%, 1.0%, 1.5%, 2.0%, 2.5%, or 3.0% w/v). These compositions can optionally also include a preservative, e.g., BAK (0.001-0.05% w/v, e.g., 0.001%, 0.0025%, 0.005%, 0.01%, 0.025%, or 0.05% w/v). In one specific example, the composition includes glycerin, polyethylene glycol (e.g., PEG400), and hypromelloses in, e.g., an amount as noted above (e.g., 0.2% v/v, 1% v/v, and 0.2% w/v, respectively).

In additional examples, the compositions include HEC (0.01-1% w/v, e.g., 0.01%, 0.025%, 0.05%, 0.07%, 0.1%, 0.5%, or 1% w/v) and/or HPMC (0.1-1% w/v, e.g., 0.1%, 0.3%, 0.5%, 0.75%, or 1% w/v, optionally in combination with dextran (e.g., dextran 70; 0.05%-1% w/v, e.g., 0.05%, 0.075%, 0.1%, 0.5%, or 1% w/v). In particular examples, these compositions can optionally include one or more preservatives such, e.g., poloxamer 407 with potassium sorbate (0.05-0.5% w/v, e.g., 0.1% w/v), BAK (0.001-0.05% w/v, e.g., 0.001%, 0.0025%, 0.005%, 0.01%, 0.025%, or 0.05% w/v), polyquad (0.0005-0.05% w/v, e.g., 0.0005%, 0.001%, 0.0025%, 0.005%, 0.01%, 0.025%, or 0.05% w/v)), or sodium perborate (e.g., 0.001-5%, e.g., 0.01-1% or 0.05-0.35%). In various specific examples, the compositions can include 0.07% w/v HEC, poloxamer 407 (e.g., 0.001-5%, e.g., 0.01-1% or 0.05-0.35%), 0.01% w/v potassium sorbate; 0.3% w/v HPMC, 0.01% w/v BAK; 0.3% w/v HPMC, 0.0002 mL 50% w/v BAK; 0.3% w/v HPMC, 0.1% w/v dextran (e.g., dextran 70); 0.3% w/v HPMC, 0.1% w/v dextran 70, 0.001% w/v polyquad; 0.3% w/v HPMC, sodium perborate.

In other examples, the compositions include PVA (0.1-3% w/v, e.g., 0.1%, 0.25%, 0.5%, 0.75%, 1.0%, 1.25%, 1.4%, 1.5%, 1.75%, 2%, 2.5%, or 3% w/v), optionally in combination with polyethylene glycol (0.1-3% w/v, e.g., 0.1%, 0.2%, 0.25%, 0.3%, 0.4%, 0.5%, 0.75%, 1.0%, 1.5%, 2.0%, 2.5%, or 3.0% w/v) and/or povidone (0.1-3% w/v, e.g., 0.1%, 0.2%, 0.25%, 0.3%, 0.4%, 0.5%, 0.6% 0.75%, 1.0%, 1.5%, 2.0%, 2.5%, or 3.0% w/v). These compositions can optionally also include a preservative, e.g., BAK (0.001-0.05% w/v, e.g., 0.001%, 0.0025%, 0.005%, 0.01%, 0.025%, or 0.05% w/v). In various specific examples, the compositions can include 1.0% w/v PVA, 1.0% v/v polyethylene glycol, and 0.01% w/v BAK; 1.4% w/v PVA and 0.6% w/v povidone; 1.4% w/v PVA and 0.005% w/v BAK; or 0.5% w/v PVA and 0.6% w/v povidone.

In further examples, the compositions can include carboxymethylcellulose (CMC; 0.1-2% w/v, e.g., 0.1%, 0.25%, 0.5%, 0.75%, 1%, 1.25%, 1.5%, 1.75%, or 2% w/v), optionally in combination with a preservative (e.g., purite, e.g., 0.001-5%, e.g., 0.01-1% or 0.05-0.35%).

In additional examples, the compositions can include sodium chloride (0.1-3% w/v, e.g., 0.1%, 0.25%, 0.5%, 0.64%, 0.75%, 0.9%, 1.0%, 1.25%, 1.4%, 1.5%, 1.75%, 2%, 2.5%, or 3% w/v), optionally in combination with a preservative, e.g., BAK (0.001-0.05% w/v, e.g., 0.001%, 0.0025%, 0.005%, 0.01%, 0.025%, or 0.05% w/v).

In further examples, the compositions can include polyvidone (1-10% w/v, e.g., 1%, 2.5%, 5%, 7.5%, or 10% w/v) or povidone (1-10% w/v, e.g., 1%, 2.5%, 5%, 7.5%, or 10% w/v), optionally in combination with a preservative, such as cetrimide (0.001-0.05% w/v, e.g., 0.001%, 0.0025%, 0.005%, 0.01%, 0.025%, or 0.05% w/v).

Other exemplary compositions include polyethylene glycol (e.g., PEG400; 0.1-2% v/v, e.g., 0.1%, 0.25%, 0.4%, 0.5%, 0.75%, 1.0%, 1.5%, or 2.0% v/v) and/or propylene glycol (0.1-2% v/v, e.g., 0.1%, 0.3%, 0.4%, 0.5%, 0.75%, 1.0%, 1.5%, or 2.0% v/v), optionally in combination with a preservative such as, for example, hydroxypropyl guar (e.g., 0.001-5%, e.g., 0.01-1% or 0.05-0.35%) and/or polyquaternium-1 (e.g., 0.001-5%, e.g., 0.01-1% or 0.05-0.35%). In one specific example, such a composition may include 0.4% v/v polyethylene glycol 400, 0.3% propylene glycol v/v, hydroxypropyl guar (e.g., 0.001-5%, e.g., 0.01-1% or 0.05-0.35%), and polyquaternium-1 (e.g., 0.001-5%, e.g., 0.01-1% or 0.05-0.35%).

In further examples, the compositions may include boric acid (0.25-4% w/v, e.g., 0.25%, 0.5%, 0.75%, 1.0%, 1.3%, 2.0%, 2.5%, 3.0%, 3.5%, or 4% w/v) and/or sodium borate (0.01-2% w/v, e.g., 0.01%, 0.05%, 0.1%, 0.32%, 0.5%, 1%, 1.5%, or 2% w/v), optionally in combination with a preservative, e.g., BAK (0.001-0.05% w/v, e.g., 0.001%, 0.0025%, 0.005%, 0.01%, 0.025%, or 0.05% w/v). A specific example of such a composition includes 1.3% w/v boric acid, 0.32% w/v sodium borate, and 0.01% w/v BAK.

In other examples, the compositions may include sodium hyaluronate (0.025-2.0% w/v, e.g., 0.025%, 0.05%, 0.1%, 0.25%, 0.5%, 0.75%, 1%, 1.25%, 1.5%, 1.75%, or 2% w/v), optionally in combination with a preservative, e.g., BAK (0.001-0.05% w/v, e.g., 0.001%, 0.0025%, 0.005%, 0.01%, 0.025%, or 0.05% w/v).

A further exemplary composition includes Hamamelis virginiana (e.g., 0.001-5%, e.g., 0.01-1% or 0.05-0.35%), optionally in combination a preservative, e.g., BAK (0.001-0.05% w/v, e.g., 0.001%, 0.0025%, 0.005%, 0.01%, 0.025%, or 0.05% w/v).

The pH of the solutions described herein can be, e.g., 6.0-8.5, e.g., 6.5-8.0, 7.0-7.8, or 7.2-7.5, as determined to be appropriate by those of skill in the art.

In various examples, solutions at or close to the normal pH of the eye (pH 7.0-7.8) are used. Examples of such compositions include the following: 0.07% HEC, poloxamer 407 (e.g., 0.001-5%, e.g., 0.01-1% or 0.05-0.35%), 0.1% potassium sorbate; 0.3% HPMC, 0.01% BAK; 0.3% HPMC, 0.1% dextran; 0.3% HPMC, 0.1% dextran 70; 0.3% HPMC, 0.1% dextran 70, 0.001% polyquad; 0.5% CMC, purite (e.g., 0.001-5%, e.g., 0.01-1% or 0.05-0.35%); 0.9% sodium chloride, 0.0002 mL 50% BAK; 5.0% povidone, 0.005% centrimide; and Hamamelis virginiana (e.g., 0.001-5%, e.g., 0.01-1% or 0.05-0.35%), 0.005% BAK.

### Methods of Treatment

TLR7 (including TLR7/8) and TLR9 agonists are administered to the eye of a subject to be treated according to the methods of the invention using methods that are known in the art for ophthalmic administration. Different routes of administration are utilized, depending upon the part of the eye to be treated. For example, for treatment of a disease or condition of the cornea, direct topical application of a formulation (e.g., as described above) to the cornea can be used, optionally in combination with a treatment used to render the cornea permeable (e.g., by the application of topical anesthetic eye drops or by mechanical abrasion or removal of corneal epithelium). For treatment of a disease or condition of another part of the eye, e.g., the retina or the choroid, a different approach to administration may be selected. For example, intravitreal or sub-retinal injection may be utilized as determined to be appropriate by those of skill in the art.

Treatment according to the methods of the invention can be carried out using regimens that are determined to be appropriate by those of skill in the art based on factors including, for example, the type of disease, the severity of disease, the results to be achieved, and the age and general health of the patient. Treatment according to the methods of the invention thus can take place just once, or can be repeated (e.g., 2, 3, 4, 5, 6, 7, 8, 9, 10, or more times). In the case of multiple treatments, appropriate intervals between treatments can be selected by those of skill in the art. The invention thus includes, e.g., hourly, daily, weekly, monthly, bi-monthly, semi-annual, or annual treatments.

The methods of the invention can be used to treat a disease or condition of the eye by preventing or reducing corneal, retinal, or choroidal neovascularization in a subject by, for example, 10% or more (e.g., at least 10%, 20%, 30%, 40%, 50%, 60%, 70%, 80%, 90%, or 100%) as compared to the amount of neovascularization observed before treatment. For example, neovascularization can be reduced by 25%, 50%, 2-fold, 5-fold, 10-fold or more, or be eliminated. Improvements in neovascularization may be assessed clinically by fundus examination and Optical Coherence Tomography (OCT) in patients, as is understood in the art.

In other examples, the methods of the invention treat a disorder or condition of the eye by reducing nerve degeneration or damage (e.g., corneal nerve damage). Nerve regeneration (e.g., recovery from nerve damage) can be enhanced by, for example, 10% or more (e.g., at least 10%, 20%, 30%, 40%, 50%, 60%, 70%, 80%, 90%, or 100%) as compared to the baseline nerve density prior to treatment. For example, nerve regeneration can be enhanced by 25%, 50%, 2-fold, 5-fold, 10-fold or more. Corneal nerve damage may be assessed visually, e.g., by in vivo confocal imaging, or by restoration of function, such as increased tear production and secretion, improved wound healing, reduced pain, improved vision, and improved reflexes, such as the corneal blink reflex.

In the case of prophylactic treatment, subjects at risk of developing a disease or condition of the eye, as described herein (e.g., subjects at risk for corneal, retinal, or choroidal neovascularization, or nerve degeneration or damage, due to a disease or condition of the eye), may be treated prior to symptom onset or when symptoms first appear, to prevent development or worsening of neovascularization, degeneration, or damage. For example, in subjects already presenting with neovascularization, further growth of vessels into presently avascular tissue can be prevented by the methods of the present invention. Similarly, in subjects already presenting with nerve damage or degeneration, further damage or degeneration can be prevented by use of the methods and compositions of the invention.

### Combination Therapies

The invention can include the use of one or more TLR7 agonist (e.g., 1, 2, 3, 4, 5, or more) and/or one or more TLR9 agonist (e.g., 1, 2, 3, 4, 5, or more). The invention can further include the use of one or more TLR7 agonist in combination with one or more TLR9 agonist. Furthermore, the invention can include combining one or more TLR7 agonist and/or one or more TLR9 agonist with one or more other agent that can be used to treat or prevent a disease or condition of the eye, such as one or more of the diseases or conditions described herein. In one example, one or more TLR7 and/or TLR9 agonist is administered in combination with pDCs. In other examples, one or more TLR7 and/or TLR9 agonists is administered with nerve growth factor (NGF). In further examples, the TLR7 and/or TLR9 agonist is administered with an anti-angiogenic agent. In addition to including methods of combination therapy, such as those described above, the invention also includes combination compositions as well as kits that include one or more agent as described herein.

Plasmacytoid Dendritic Cells (pDCs)

According to the methods of the invention, the administered TLR7 and/or TLR9 agonist is used to stimulate resident pDCs of the eye. In addition to this, treatment may be bolstered by the co-administration of pDCs, as determined to be appropriate by those of skill in the art.

pDCs circulate in the blood and can also be found in peripheral lymphoid organs. They are bone marrow-derived innate immune cells that express Toll-like receptors (TLR) 7 and 9. In mice, they express low levels of CD11c, which differentiates them from conventional dendritic cells (cDCs), and exhibit PDCA-1, Siglec-H, and CD45R/B220. In humans, pDCs are positive for blood-derived dendritic cell antigen (BDCA)-2 (CD303), BDCA-4 (CD304), and CD123. Upon activation, they produce large amounts of type 1 interferons (see, e.g., Tversky et al., Clin. Exp. Allergy 38(5):781-88, 2008; Asselin-Paturel et al., Nat. Immunol. 2(12):1144-50, 2001; Nakano et al., J. Exp. Med. 194(8):1171-8, 2001; Bjorck, Blood 98(13):3520-6, 2001).

pDCs for use in the invention can be isolated from a subject to whom they are to be administered or they can be obtained from a donor (e.g., a human donor). pDCs can be isolated from blood or bone marrow using standard techniques including, e.g., density gradient centrifugation and marker-based cell separation. Optionally, the pDCs can be cultured and/or frozen prior to use. Furthermore, the pDCs can be obtained by the stimulation of cultured bone marrow cells. For example, peripheral blood mononuclear cells (PBMCs) can be isolated from blood using, e.g., Ficoll gradient density centrifugation. Then, pDCs can be isolated from PBMCs based on a pDC-specific or pDC-enriched marker (e.g BDCA-2, BDCA-4, or CD123). An antibody against such a marker (e.g., an anti-BDCA-2, anti-BDCA-4, or anti-CD123 antibody) can be used in this isolation step using standard methods (e.g., microbead or magnetic bead-based separation or fluorescence-activated cell sorting [FACS]).

In a specific example, 5-10 ml blood is collected from a subject via routine venipuncture and is placed in a tube containing citrate as an anti-coagulant. Next, PBMCs are separated by standard Ficoll density gradient centrifugation. After isolating PBMCs, pDCs are selected via commercially available magnetic beads according to the manufacturer's instructions (Miltenyi Biotec). In brief, PBMCs are blocked with an anti-Fc receptor antibody for 15 minutes at room temperature (RT). Next, samples are labeled by incubation with an anti-BDCA2 antibody conjugated with microbeads for 30 minutes at 4° C. Cells labeled with magnetic bead-conjugated BDCA-2 antibodies (which will constitute pDCs) are then applied to a separation column, placed in a separation device standing on a magnetic field. By washing the separation column with sterile washing buffer, BDCA2-negative cells (non-pDCs) are washed out, while BDCA-2⁺ labeled pDCs stay attached to the column. At this step, the separation column is removed from the magnetic field and pDCs are eluted by pushing washing buffer through the column. After separation, the number of pDCs is determined by routine Trypan blue staining on a portion of collected cells and the purity of the sample is measured by immunofluorescence staining with a BDCA2 fluorochrome-conjugated antibody (as well as other human pDC markers including BDCA-4 and CD123, if needed) and analyzed with FACS. In case analysis shows not satisfactory purity of the isolated cells (e.g., less than 85%), purity can be improved by another round of magnetic separation. Cells are then centrifuged and resuspended in sterile saline or tissue glue for adoptive transfer purposes.

Accordingly, compositions including pDCs and a pharmaceutically acceptable carrier or diluent can be used in the methods of the present invention. For example, pDCs prepared, e.g., as described above, can be diluted or concentrated to a final concentration of, e.g., 10⁴-10⁸, 10⁶-10⁷, or 10⁶ cells per ml in a pharmaceutically acceptable carrier or diluent. The desired concentration of cells will vary depending on the method of administration and the type and severity of the disease or condition being treated. Depending upon the particular application, the carrier or diluent can be selected from, e.g., liquids, creams, drops, or ointments, as can be determined by those of skill in the art. For example, the cells can be administered by the use of a tissue adhesive or glue, such as a biologic adhesive (e.g., a fibrin-based adhesive or glue, such as Tisseel). Alternatively, a solution may be used (e.g., phosphate buffered saline, sterile saline, or sterile culture medium (e.g., RPMI or DMEM)). The compositions used in the invention typically include pDCs are at least 50% (e.g., at least 60%, 75%, 90%, 95%, 99%, or 100%) of the cells present in the compositions.

In one example, isolated pDCs are diluted in tissue glue (e.g., Tisseel) at a density of about 10⁶ cells/μl and applied to the cornea. If the corneal epithelium is not intact, the cells can be applied directly onto the cornea, but if the corneal epithelium is intact, it can be treated to make it permeable prior to administration of the cells. This can be achieved, for example, by the application of topical anesthetic eye drops or by mechanical abrasion or removal of corneal epithelium.

For treatment of a disease or condition of another part of the eye, e.g., the retina or the choroid, a different approach to administration may be selected. For example, intravitreal or sub-retinal injection may be utilized as determined to be appropriate by those of skill in the art. In a specific example, isolated pDCs are diluted in sterile culture media or phosphate buffered saline at a concentration of about 10⁶ cells/μl and administered to the retina or choroid by routine intravitreal or sub-retinal injection.

The following non-limiting examples are illustrative of the present disclosure.

### EXAMPLES

### Example 1: TLR7 Agonists—Nerve Regeneration

In this study, we explore neurotrophic properties of pDCs. We demonstrated that pDCs dwell in close spatial proximity of corneal sub-basal nerve plexus and their depletion is accompanied by degeneration of corneal nerves and abrupt dampening of corneal sensory nerve function. Further, we show that pDC promote nerve regeneration and neurite outgrowth in vitro and in vivo in part through secretion of nerve growth factor (NGF). Also, we describe that stimulating pDCS through TLR7 can enhance secretion of NGF and consequently promote neurite outgrowth via phosphorylating cAMP response element-binding protein (CREB) by p38 mitogen-activated protein (MAP) kinase.

### Results

### Plasmacytoid Dendritic Cells Reside in Close Proximity of Sub-Basal Nerve Plexus in Normal Cornea

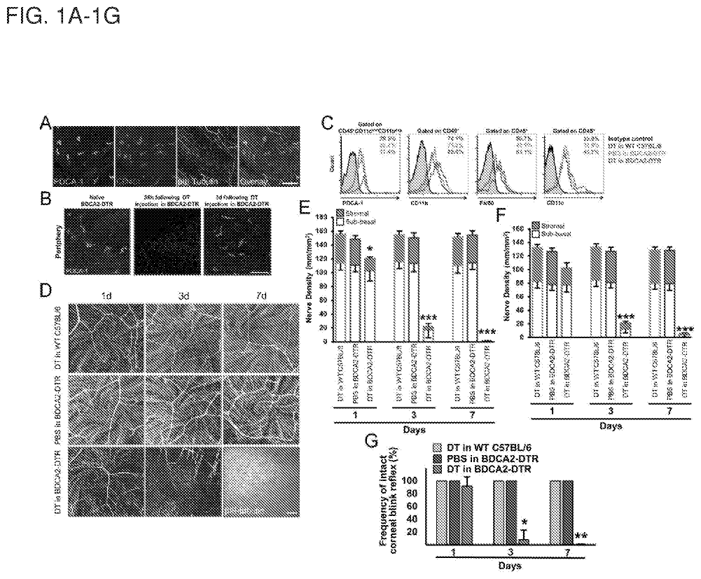

We have recently showed that cornea hosts resident pDCs under steady state. In order to study potential communication of pDCs with corneal nerves, we first assessed spatial relation of pDCs and corneal nerves. As shown in FIG. 1A, whole mount immunofluorescence (IF) staining of naïve wild-type (WT) C57BL/6 cornea with βIII-tubulin (pan-neuronal marker; white), CD45 (pan-leukocyte marker; red), and PDCA-1 (pDC marker; green), reveals that pDCs lay in anterior stroma in close proximity of corneal sub-basal nerve plexus.

### Local Depletion of Corneal Plasmacytoid Dendritic Cells is Accompanied by Abrupt Corneal Nerve Loss

Next, we depleted resident corneal pDCs by subconjunctival (subconj.) injection of 30 ng Diphtheria toxin (DT) in transgenic BDCA2-DTR mice. In these mice, Diphtheria toxin receptor is expressed under transcriptional control of human BDCA2, a specific pDC gene. Therefore, in these transgenic mice specifically pDCs are ablated upon exposure to DT (Swiecki et al., Immunity 33(6):955-66, 2010). As shown in FIG. 1B-C, we observed that our method of local depletion of pDCs in cornea of BDCA2-DTR mice selectively ablates pDCs, sparing other populations of bone marrow derived cells (CD11b⁺, F4/80⁺, and CD11c^{moderate/high} cells). Also, we have previously shown that although single injection of DT is successful in depleting about 80-90% of resident corneal pDCs, these cells are quickly repopulated in 3 days following injection (FIG. 1B). Thus, we repeated subconj. DT injections every 48 hours to keep cornea devoid of pDCs.

Upon pDC depletion, we assessed corneal blink reflex and subsequently nerve density on excised corneal whole-mounts by immunofluorescence staining followed by confocal microscopy. As shown in FIGS. 1D-F, we observed that pDC depletion is accompanied by severe degeneration of sub-basal and stromal nerve plexuses as early as day 1 following pDC depletion in both center (103.1±15.3 mm/mm² in sub-basal plexus and 17.1±2.4 in stromal plexus) and periphery (77.4±10.6 in sub-basal plexus and 24.6±8.0 in stromal plexus) of the cornea. Notably, degeneration of corneal nerves progressed during the course of experiments; as 7 day following pDC depletion, almost all corneal nerves in the center (1.1±0.7 mm/mm² in both plexuses combined) and periphery (5.3±3.9 in both plexuses combined) of cornea were degenerated. In order to assess possible contribution of administration of DT or subconj. injection in BDCA2-DTR mice, we used two control groups in these experiments: WT C57BL/6 mice receiving subconj. 30 ng DT and BDCA2-DTR mice treated with similar volume of PBS. Notably, we did not observe any alterations in corneal nerve density following subconj. injection of PBS or DT in BDCA2-DTR and WT C57BL/6 mice, respectively. In agreement with this finding, we observed that corneal blink reflex is diminished in the center of cornea upon pDC depletion (8.3% positive blink reflex on day 3, and 0% on day 7), however, this reflex remains intact in two control groups (frequency of positive blink reflex: 100%, p<0.001; FIG. 1G).

### Repopulation of Plasmacytoid Dendritic Cells Promotes Corneal Nerve Regeneration

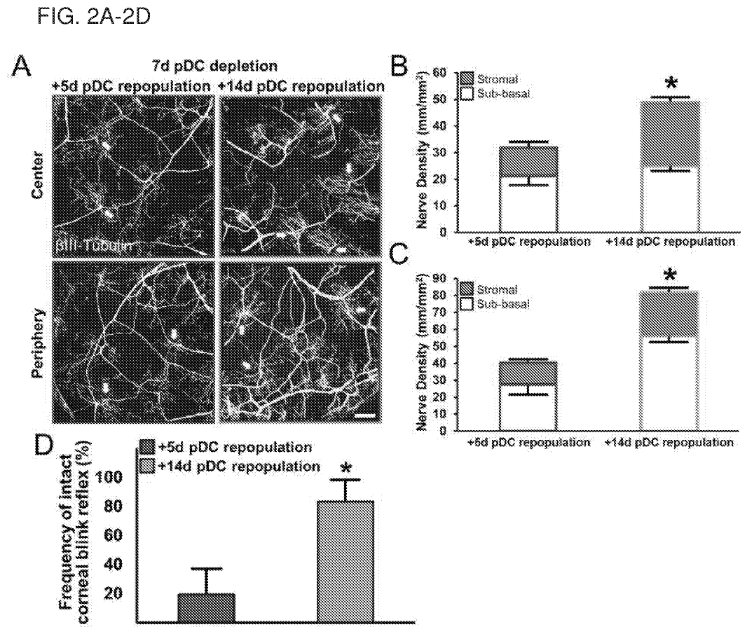

Next, in order to study if pDCs can induce nerve regeneration, we assessed corneal nerve regeneration after initial degeneration. For this experiment, we initially depleted pDCs in the cornea of BDCA2-DTR mice for 7 days to induce nerve degeneration; next, we stopped DT injection to let pDCs repopulate in the cornea. 5 and 14 days following stopping DT injection, we measured corneal sub-basal and stromal nerve densities and observed substantial progressive regeneration of both plexuses in the center (31.8± on day 5 vs. 49.0± on day 14, p<0.001) and periphery (40.5± on day 5 vs. 81.8± on day 14, p<0.001) of cornea upon pDC repopulation (FIG. 2A). In line with this finding, we observed that 5 days after repopulation of cornea, corneal blink reflex is re-established in 19.3% of the mice and by day 14 following pDC repopulation, 93% of mice exhibit normal blink reflex (p<0.001; FIG. 2B).

### Plasmacytoid Dendritic Cells are Vital Source of Nerve Growth Factor in Cornea

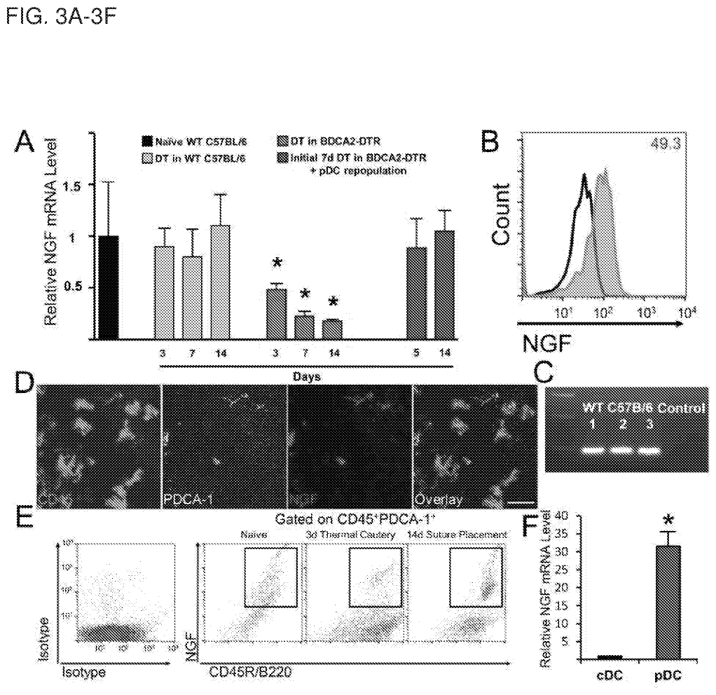

Furthermore, we studied the molecular mechanism orchestrating this observation. Considering numerous reports on the necessity of nerve growth factor (NGF) in maintenance and regeneration of peripheral nerves, we assessed the mRNA level of this neurotrophic molecule via semi-quantitative real-time PCR (qRT-PCR) in corneal stroma, where pDCs reside, upon pDC depletion (Finn et al., J. Neurosci. 20(4):1333-41, 2000; Patel et al., Neuron 25(2):345-57, 2000; White et al., J. Neurosci. 16(15):4662-72, 1996). As illustrated in FIG. 3A, we observed that NGF level are decreased in cornea following pDC depletion in BDCA2-DTR mice (0.18±0.02-fold change on day 7 following pDC depletion, p<0.001), however, its level reaches the levels of the steady state in naïve WT C57BL/6 mice following pDC repopulation.

Next, in order to assess if pDCs may present a source of NGF, we initially took advantage of transgenic DPE-GFP×RAG1^{-/-} mouse, with specifically GFP-tagged pDCs (Iannacone et al., Nature 465(7301):1079-83, 2010; Iparraguirre et al., J. Leukoc. Biol. 83(3):610-20, 2008). As shown in FIG. 3B, sorted splenic GFP-tagged pDCs of naive DPE-GFP×RAG1^{-/-} mice, were stained with NGF, suggesting pDCs may serve as source of NGF. Next, in order to confirm that pDCs express NGF, we assessed presence of NGF mRNA in sorted GFP-tagged pDCs by reverse transcriptase PCR followed by PCR using NGF primer. Next, PCR products from the samples as well as controls lacking template RNA in cDNA synthesis step were subjected to agarose gels electrophoresis. As shown in FIG. 3C, sorted GFP-tagged pDCs from the spleen naïve DPE-GFP×RAG1^{-/-} mice harbor endogenous NGF mRNA.

Further, we analyzed if corneal pDCs can also produce NGF similar to splenic pDCs. As depicted in FIG. 3D, confocal micrograph of WT C57BL/6 mice showed that pDCs (CD45⁺PDCA-1⁺) co-stain with NGF (red) in the normal cornea. In order to validate this finding, we performed flow cytometry on single cell suspension of digested naïve as well as inflamed corneas of WT C57BL/6 mice. For induction of inflammation, we applied corneal thermal cautery burn and suture placement, both of which are well-known techniques for sterile inflammation in cornea (Cursiefen et al., Proc. Natl. Acad. Sci. U.S.A. 103(3):11405-10, 2006; Streilein et al., Invest. Ophthalmol. Vis. Sci. 37(2):413-24, 1996; Williamson et al., Invest. Ophthalmol. Vis. Sci. 28(9):1527-32, 1987). We observed that corneal pDCs (identified by expression of CD45, PDCA-1, and B220) also co-stain with NGF in steady state as well as upon inflammation (FIG. 3E). Notably, in order to assure identifying pDCs accurately, we used two markers for pDCS (PDCA-1 and B220) in this experiment, as previous reports suggest that use of PDCA-1 as a single marker for identification of pDCs may encompass other cell entities including B cells, plasma cells, rare population of cDCs, as well as other immune cells, in particular following inflammation (Bao et al., Eur. J. Immunol. 41(3):657-68, 2011; Blasius et al., J. Immunol. 177(5):3260-5, 2006; Vinay et al., J. Immunol. 184(2):807-15, 2010).

We also compared the NGF mRNA content of splenic pDCs with cDCs; we sorted GFP-tagged pDCs from the spleen of naïve DPE-GFP×RAG1^{-/-} and cDCs for WT C57BL/6 mice and compared their relative NGF mRNA levels via qRT-PCR. Distinguishably, we observed remarkably higher levels of NGF in pDCs (31.5±4.1 folds more, p<0.01) versus in their classic counterparts (FIG. 3F).

Plasmacytoid Dendritic Cells Enhance Neurite Outgrowth in Trigeminal Ganglion Cell Culture Through Secretion of Nerve Growth Factor In Vitro

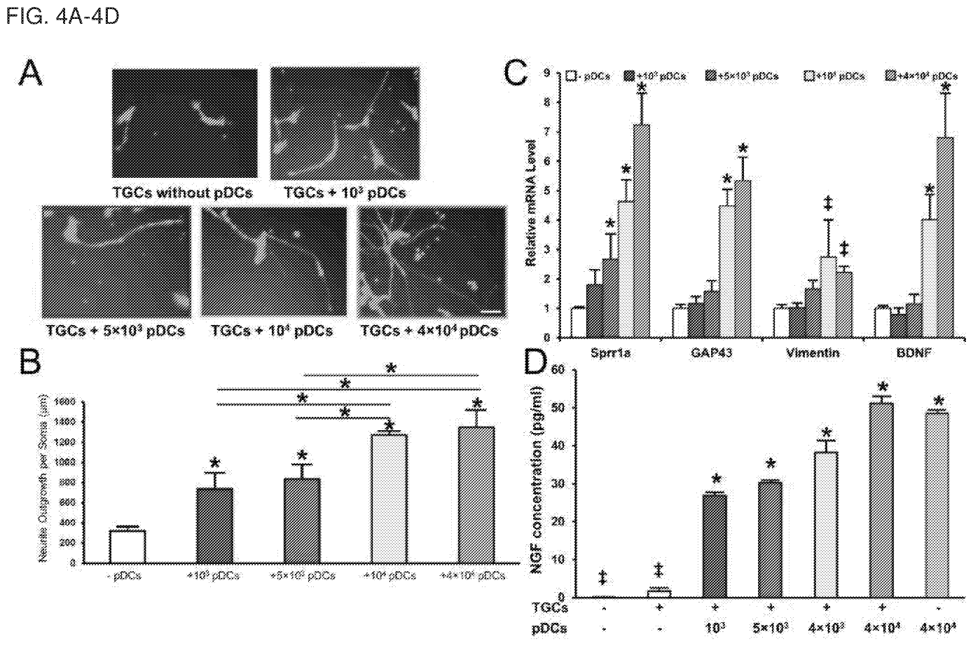

We further assessed if pDCs secret functionally active NGF. We cultured isolated trigeminal ganglion cells (TGCs) for one day and then added different numbers of sorted splenic GFP-tagged pDCs from naïve DPE-GFP×RAG1^{-/-} mice to transwells to conduct a co-culture study. We assessed neurite outgrowth on TGCs, 3 days after co-culture and observed a considerable increase in the length of TGC neurites in parallel to density of pDCs in transwells (FIGS. 4A and 4B).

To further confirm our finding, we also measured expression of neuro-regenerative markers including small proline-rich repeat protein 1a (Sprr1a), growth-associated protein-43 (Gap-43), vimentin, and brain derived neurotrophic factor (BDNF) in cultured TGCs (Pernet et al., Cell Death Differ. 19(7):1096-108, 2012; Bonilla et al., J. Neurosci. 22(4):1303-15, 2002; Sun et al., Nature 480(7377):372-5, 2011; Sarkar et al., Invest. Ophthalmol. Vis. Sci. 54(9):5920-36, 2013). As demonstrated in FIG. 4C, in line with our neurite outgrowth measurement, we observed higher expression of neuro-regenerative markers in cultured neurons along with increase in the density of pDCs in the system.

Next, to study if pDCs secret NGF in vitro, we measured the level of NGF in the co-culture media. We noted substantial increase in the amount of NGF in the media in conditions of culturing TGCs with pDCs versus TGCs alone. Interestingly, the increase in NGF was dependent on pDCs as similar amounts of NGF were detected in the cell culture media of pDC and TGC co-culture versus pDC monoculture in transwells (FIG. 4D).

Enhanced Neurite Outgrowth Induced by Plasmacytoid Dendritic Cells in not Solely Dependent on Nerve Growth Factor

Having found that pDCs promote TGC neurite outgrowth, we planned to assess if solely secretion of NGF mediates this process. Therefore, we performed another set of pDC and TGC co-culture experiments with application of anti-NGF blocking antibody. Considering the large number of pDCs needed for these experiments, we boosted production of pDCs in mice by injecting FMS-like tyrosine kinase 3 ligand (Flt3L)-secreting melanoma cell line 10-12 days prior to pDC isolation to overcome the rarity of pDCs, as previously described (Mora et al., Nature 424(6944):88-93, 2003). Flt3L cytokine signaling is in particular essential for expansion and maturation of pDCs, and to a lesser extent cDCs (Schmid et al., Immunol. Rev. 234(1):32-44, 2010; Waskow et al., Nat. Immunol. 9(6):676-83, 2008; Eidenschenk et al., Proc. Acad. Natl. Acad. Sci. U.S.A. 107(21):9759-64, 2010). Thus, promoting pDC development by Flt3L has been widely used to isolate pDCs from mice (Bjorck et al., Blood 98(13):9520-6, 2001; Brawand et al., J. Immunol. 169(12):6711-9, 2002; Gilliet et al., J. Exp. Med. 195(7):953-8, 2002; Asselin-Paturel et al., J. Immunol. 171(12):6466-77, 2003; Naik et al., Methods Mol. Biol. 595:167-76, 2010).

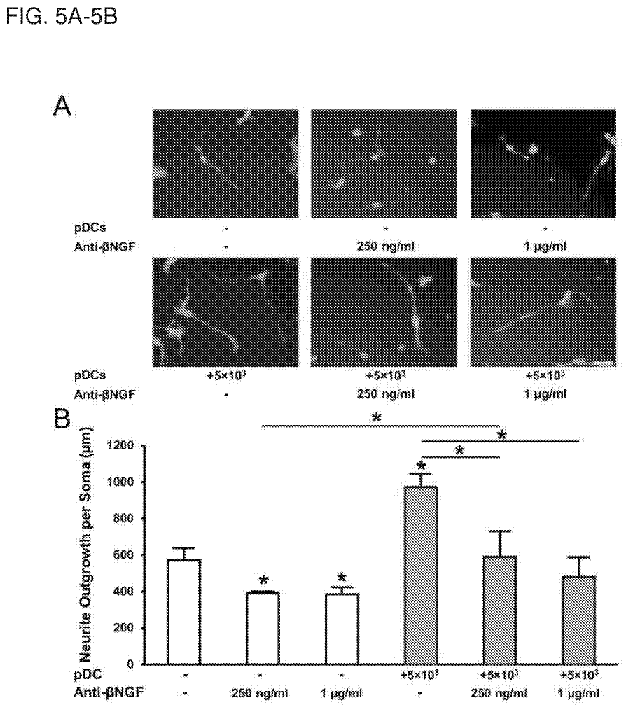

We co-cultured TGCs and pDCs for 5 days under two concentrations of anti-NGF blocking antibody. Similar to our previous experiment, we detected longer neurites when culturing TGCs with pDCs (FIGS. 5A and 5B). Blocking NGF reduced neurite outgrowth in both conditions of culturing TGCs with or without pDCs. Notably, although we observed reduced neurite outgrowth after blocking NGF with 250 ng/ml anti-NGF, neurite length was still greater when pDCs were present in co-culture compared to culturing TGCs alone. This observation suggests that factors other than NGF may also play a role in pDC-induced enhanced neurite outgrowth in vitro. Nevertheless, we did not observe differences in neurite length when blocking NGF with 1 μg/ml anti-NGF in conditions with and without pDCs. This can be explained by possible cross-binding of anti-NGF blocking antibody with other neurotrophic molecules including BDNG, glial cell line-derived neurotrophic factor (GDNF), neurotrophin 3, and neurotrophin 4/5 at extremely high concentration of 1 μg/ml.

Stimulation of Plasmacytoid Dendritic Cells Through TLR7 Enhances their Neurotrophic Properties

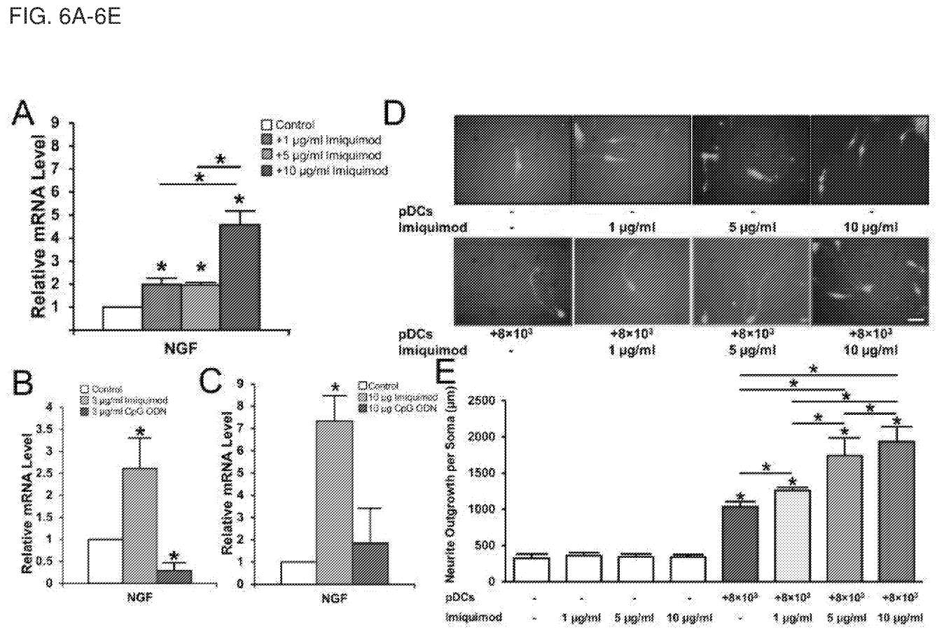

We next assessed if synthesis and secretion of NGF can be enhanced in pDCs. We sorted splenic GFP-tagged pDCs from DPE-GFP×RAG1^{-/-} mice 10-14 days following receiving Flt3L secreting melanoma cells and cultured them for 3 days under different concentrations of specific TLR7 agonist, Imiquimod (Lee et al., Proc. Natl. Acad. Sci. U.S.A. 100(11):6646-51, 2003; Miller et al., Intl. J. Immunopharmacol. 21(1):1-14, 1999). As illustrated in FIG. 6A, we observed a dose dependent increase in NGF relative mRNA level following treating splenic pDCs with Imiquimod.

Next, in order to extend our finding of enhanced NGF expression by splenic pDCs through stimulation of TLR7, we assessed the effect of activating corneal pDCs on NGF mRNA level. Hence, we sorted GFP-tagged pDCs from digested corneas of naïve DPE-GFP×RAG1^{-/-} mice (n=8-10) and cultured them for 24 hours under treatment with Imiquimod, CpG 1826 oligonucleotide (CpG-ODN; TLR9 agonist), or control non-CpG 1826 oligonucleotide ODN (control ODN) (Heikenwalder et al., Nat. Med. 10(2):187-92, 2004; Krogmann et al., PLoS One. 11(1):e0146326, 2016). We then performed single cell PCR on cultured corneal pDCs. FIG. 6B shows that, in agreement with our previous experiment, stimulation of corneal pDCs with TLR7 agonist increases relative mRNA level of NGF (2.6±0.7-fold increase, p<0.001); nevertheless, stimulation with TLR9 did not result in increased NGF mRNA transcription in corneal pDCs (FIG. 6B).

Further, we assessed the effects of pDC activation with TLR7 and 9 agonists on NGF production in vivo. For this experiment, we inoculated 10 μg Imiquimod, CPG ODN, or control ODN to the cornea of DPE-GFP×RAG1^{-/-} mice and 24 hours later, we euthanized the animals, excised and digested the corneas, and sorted GFP-tagged pDCs. Single cell PCR on sorted pDCs validated our in vitro findings, by showing remarkable increase in relative mRNA levels of NGF following administration of TLR7 agonist (7.3±1.1-fold increase, p<0.001), but not TLR9 agonist (1.8±1.6 fold increase, p=0.21; FIG. 6C).

We then assessed if stimulating pDCs through TLR7 leads to production of functionally active NGF. Therefore, we mono-cultured TGCs and co-cultured TGCs with pDCs under different concentrations of Imiquimod. We observed that while TLR7 agonist does not significantly enhance neurite outgrowth in TGC mono-culture, it enhances neurite outgrowth induced by pDCs in pDC and TGC co-culture in vitro (FIGS. 6D and 6E).

Enhanced Production of NGF Upon Stimulation of Plasmacytoid Dendritic Cells with TLR7 Agonist is Mediated Through Phosphorylion of CREB Via p38 MAP Kinase

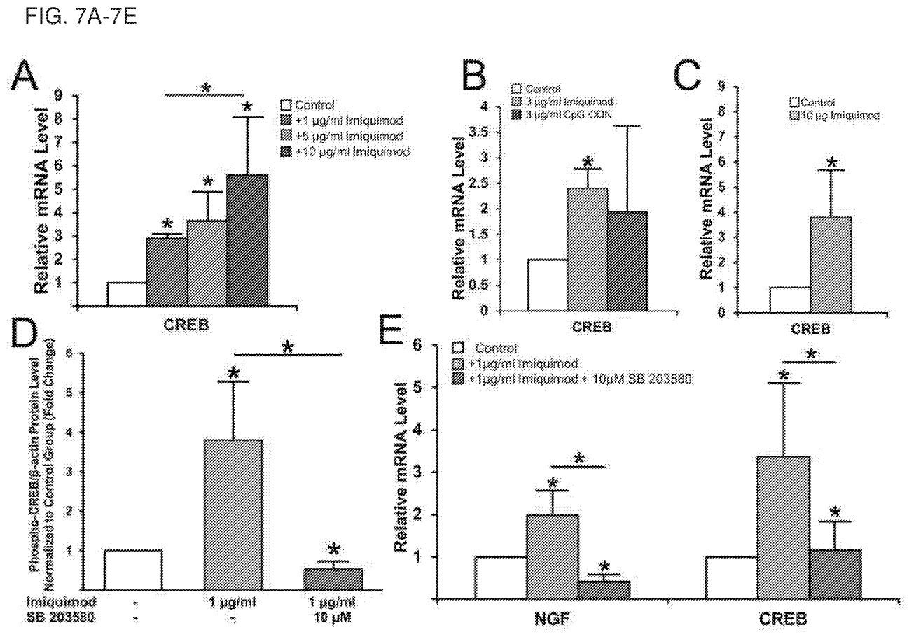

It is well established that CREB transcription factor plays an important role in the functions of neurotrophic molecules (Finkbeiner et al., Neuron 19(5):1031-47, 1997). Therefore, in order to study the molecular mechanism mediating the increase in NGF mRNA following stimulating pDCs through TLR7, we assessed relative mRNA level of CREB following treating splenic GFP-tagged pDCs with Imiquimod. As depicted in FIG. 7A, a dose-dependent increase in CREB mRNA was evident following treating splenic pDCs with Imiquimod.

Similar to experiments described above, we next assessed if neurotophic properties of corneal pDCs parallels splenic pDCs upon stimulation with TLR7 or 9 agonists. Therefore, we isolated GFP-tagged corneal pDCs and stimulated them with Imiquimod, CpG ODN, or control ODN for 24 hours in vitro. Remarkably, we observed that while treatment of corneal pDCs with TLR7 agonist increases transcription of CREB in vitro, administration of TLR9 agonist, CpG-ODN, does not result in significant escalation in CREB transcription (FIG. 7B). We further aimed to evaluate if application of Imiquimod in vivo alters CREB mRNA level. As illustrated in FIG. 7C, consistent with our in vitro culture, GFP-tagged pDCs isolated from the corneas of GFP×RAG1^{-/-} mice undergoing ocular inoculation of 10 μg Imiquimod showed higher CREB mRNA levels compared to control inoculation.

Next, to further explore the mechanism of NGF expression in pDCs following pDC stimulation with TLR7 agonist, we studied if p38 MAP kinase plays a role in this process. In this regard, we isolated GFP-tagged pDCs from the spleen of DPE-GFP×RAG1^{-/-} mice 10-14 days following subcutaneous administration of Flt3L secreting melanoma cells. Isolated cells were cultured and pre-treated with 10 μM SB 203580, a pyridinyl imidazole which selectively inhibits p38 MAP kinase or PBS (Cuenda et al., FEBS Lett. 364(2):229-33, 1995). After 1 hour, cells were treated with 1 μg/ml Imiquimod or PBS. 3 days later, we assessed phosphorylation of CREB in the cultured pDC lysates and as well as relative NGF mRNA level. We observed that stimulation of pDCs with TLR7 agonist, enhances phosphorylation of CREB (3.8±1.5-fold increase, p<0.001), however, inhibiting p38 MAP kinase, prevents this process (0.5±0.2 fold change in comparison to control, p<0.001; FIG. 7D). Similarly, we observed that inhibition of p38 MAP kinase precludes expression of NGF induced by TLR7 agonist (2.0±0.6 fold increase upon treatment with Imiquimod vs. 0.4±0.1 fold change by prior inhibition of p38 MAP kinase, p<0.001) (FIG. 6E), suggesting that increase in NGF following stimulation of pDCs through TLR7 is dependent on p38 MAP kinase activity and phosphorylation of CREB. Notably, repeating the latter experiments with pDCs sorted from the spleen of WT C57BL/6 mice undergoing injection of Flt3L secreting melanoma cells 10-14 days prior to pDC isolation, we observed comparable results.

### Experimental Procedures

### Animals

Six- to ten-week-old male wild-type (WT) C57BL/6 mice were purchased from Charles River (Charles River Laboratories International, Wilmington, Mass.); DPE-GFP×RAG1^{-/-} mice, with specifically GFP-tagged pDCs, and BDCA2-DTR mice (Iannacone et al., Nature 465(7301):1079-83, 2010; Iparraguirre, J. Leukoc. Biol. 83(3):610-20, 2008). C57BL/6 background; Jackson Laboratory, Bar Harbor, Me.) were bred in house in specific pathogen free conditions. BDCA2-DTR mice were bred into homozygous for the experiments (For culturing TGCs, 10-day old transgene negative pups were used (Swiecki et al., Immunity 33(6):955-66, 2010). All protocols were approved by Schepens Eye Research Institute and Tufts Medical Center and Tufts University School of Medicine Animal Care and Use Committees (IACUC) and animals were treated according to the Association for Research in Vision and Ophthalmology (ARVO) Statement for the Use of Animals in Ophthalmic and Vision Research.

### Assessment of Corneal Sensation

Corneal blink reflex was assessed as previously described (Yamaguchi et al., PLoS One 8(8):e70908, 2013). In brief, an 8-0 nylon thread was applied to the central cornea of un-anesthetized mice under direct vision through a dissecting microscope to avoid contact with whiskers and eyelashes. The procedure was repeated three times on each mouse to ensure reproducibility.

### Corneal Immunofluorescence Staining, Confocal Microscopy, and Image Quantification

For immunofluorescent staining with NGF, corneal epithelium was removed by fine forceps following incubating corneas with 20 mM EDTA (Sigma-Aldrich, St. Louis, Mo.) at 37° C. for 30 minutes, as previously described (Hamrah et al., Invest. Opthalmol. Vis. Sci. 43(3):639-46, 2002). Excised whole corneas or corneal stromas were fixed with chilled acetone (Sigma-Aldrich) at −20° C. for 15 minutes. After washing fixed samples with PBS for 3 times, samples were blocked in 2% bovine serum albumin (BSA; Sigma-Aldrich) and 1% anti-CD16/CD32 Fc receptor (FcR) mAb (2.4G2; Bio X Cell, West Lebanon, N.H.) for 30 minutes at RT. Next, samples were stained with fluorophore-conjugated CD45 (BioLegend, San Diego, Calif.), PDCA-1 (Miltenyi Biotec, Bergisch Gladbach, Germany), βIII-Tubulin (R&D Systems, Minneapolis, Minn.), or biotinylated anti-NGF (BioLegend) antibodies overnight at 4° C. Following three washes with PBS, if needed, samples were incubated with secondary anti-biotin antibody (BioLegend), for 1 hour at RT. Next, after washing with PBS for 3 times, corneas were mounted with Vectashield with DAPI (Vector Labs, Burlingame, Calif.) and underwent microscopy via upright TCS SP5 Leica confocal microscope (Leica Microsystems, Germany). For quantification purposes, 3 images from periphery and a single image was taken the center of cornea. Quantification of nerve density was performed via NeuronJ plugin for ImageJ software (NIH, Bethesda, Md.), as previously described (Meijering et al., Cytometry A 58(2):167-76, 2004; Yamaguchi et al., PLoS One 8(8):e70908, 2013; Hu et al., PLoS One 10(9):e0137123, 2015).

### Corneal Single Cell Suspension and Flow Cytometry

Cornea was digested to yield single cells as previously described (Hamrah et al., Invest. Ophthalmol. Vis. Sci. 44(2):581-9, 2003). In brief, naïve and inflamed corneas were excised (n=12 for naïve and n=5 for inflamed groups), cut into small pieces, and digested with 2 mg/ml collagenase D (Roche, Indianapolis, Ind.) and 0.05 mg/ml DNAse (Roche, Indianapolis, Ind.) for 45 minutes at 37° C. in a humidified atmosphere with 5% CO2. Next, digested corneas passed through a 40 mm cell strainer (BD Falcon, Becton-Dickinson, Franklin Lakes, N.J.) to remove undigested materials. Then, single corneal cells were washed, blocked with 1% anti-CD16/CD32 FcR mAb (Bio X Cell) in 0.5% BSA containing 0.5% Tween 20 (Sigma-Aldrich) for 20 minutes at RT, and stained with combinations of antibodies against CD45, CD11c, CD11b, F4/80, PDCA-1, CD45R/B220, NGF, or their respective isotype controls (all BioLegend except for CD11c, from BD Bioscience, San Jose, Calif.) for 30 minutes in FACS buffer at RT in the dark. After washing with PBS, samples were incubated with secondary antibody against biotin (Jackson ImmunoResearch Laboratories, Inc., West Grove, Pa.) for 30 minutes at RT. Afterwards, samples were washed and reconstituted in 4% paraformaldehyde and underwent data acquisition with a BD LSR II flow cytometer (BD Biosciences). Data were analyzed with FlowJo V9.2 (FlowJo, LLC, Ashland, Oreg.). Forward and side scatter plots were used to exclude dead cells, debris, and doublets.

### Splenic pDC and cDC Isolation

pDCs were isolated from DPE-GFP×RAG1^{-/-} and cDCs were purified from naïve WT C57BL/6 mice, respectively. DPE-GFP×RAG1^{-/-} mice underwent subcutaneous injection of 5×10⁶ B16 murine Flt3L-secreting melanoma tumor cells. 10-14 days later, mice were euthanized; spleens were harvested, and mechanically disturbed using a 5 ml syringe plunger and were filtered through a 40 mm cell strainer (BD Falcon). Next, after incubation with ice-cold ammonium chloride (ACK) lysis buffer (Biofluids, Rockville, Md.) for 1 minute to remove contaminating RBCs, cells were washed with PBS. GFP-tagged pDCs were sorted via Moflo Cell Sorter (Beckman Coulter, Brea, Calif.). For obtaining pDCs for co-culturing TGCs with different densities of pDCs, as well as for flow cytometry and presence of NGF mRNA on GFP-tagged splenic pDCs, and comparing the NGF mRNA content in pDCs and cDCs, naïve DPE-GFP×RAG1^{-/-} mice were used. cDCs were purified using CD11c MicroBeads (Miltenyi Biotec) according to the manufacturer's instruction, with a purity of more than 85%.

### pDC Culture

Isolated splenic pDCs were cultured in 48 well plates (200,000 cells/well) for 3 days with Ham's F-12 Nutrient Mix (Gibco, Carlsbad, Calif.) supplemented with 10% heat inactivated fetal bovine serum (FBS; Gemini Bioproducts, Woodland, Calif.), and 1% penicillin/streptomycin (Life Technologies, Carlsbad, Calif.). Cells were treated with 1, 5, and 10 μg/ml Imiquimod (InvivoGen, San Diego, Calif.) or PBS upon seeding. In another set of experiments, cells were pretreated with 10 μM SB 203580 (p38 MAP kinase inhibitor; Sigma-Aldrich) or PBS 1 hour prior to addition of 1 μg/ml Imiquimod (InvivoGen) or PBS. Cells were harvested for RNA or protein extraction 3 days after culture.

### pDC and TGC Co-Culture and Microscopy

Initially, 10-day old pups were euthanized, TGs were excised, chopped into small fragments, and digested in 2 mg/ml Collagenase D (Roche), 2 mg/ml DNAse I (Roche), and 5 mg/ml Dispase II (Sigma-Aldrich) in Hank's Balanced Salt Solution (Gibco) at 37° C. for 30 minutes. Next, after filtering, cells were layered over a 12.5% on 28% Percoll (GE Healthcare, Pittsburgh, Pa.) gradient in L15 media (Gibco) and centrifuged at 1300 g for 10 minutes. Following removing debris in the percoll interface, purified TGCs were recovered from the bottom of the gradient. Next, 10,000 cells/well were seeded in 24 well cell culture plates coated with growth factor reduced Matrigel (Corning Inc, Corning, N.Y.) in Ham's F-12 Nutrient Mix (Gibco) supplemented with 10% heat inactivated FBS (Gemini Bioproducts), 1% penicillin/streptomycin (Life Technologies) and 100 ng/ml NGF (Sigma-Aldrich). After one day of culture, media was changed to a similar media without NGF and sorted pDCs with different numbers were added to transwells. On day 3 following co-culture, transwells were removed, TGCs were stained with 1 μM Calcein (Life Technologies) and underwent imaging by an inverted Nikon Eclipse Ti inverted microscope (Nikon Inc., Melville, N.Y.) equipped with an Andor Clara E digital camera (Andor Technology Ltd., Belfast, UK). Three images were taken from each well. Further, cell culture media was collected and kept at −80° C. for further protein measurement. TGCs were used for RNA extraction and quantitative real-time PCR.

In another set of experiments, TGCs (10,000 cells/well) were cultured one day prior to adding sorted pDCs (5,000 cells/well); afterwards, cells were treated with PBS, 250 ng/ml, or 1 μg/ml β-NGF blocking antibody (Biosensis, Thebarton, Australia). On day 5 following co-culture, TGCs were stained with 1 μM Calcein and imaged.

In another set of experiments, TGCs (10,000 cells/well) were cultured one day to adding 8,000 pDCs/well) to transwells. On day 1 of co-culture, cells were treated PBS, 1, 5, or 10 μg/ml Imiquimod (InvivoGen); on day 3 following co-culture, transwells were removed and TGCs were stained with 1 μM Calein and imaged. All experiments were performed in triplicate and data represent three independent experimentations. Neurite outgrowth was measured NeuronJ plugin (Meijering et al., Cytometry A 58(2):167-76, 2004) for ImageJ software (NIH, Bethesda, Md.).

RNA Isolation, cDNA Synthesis, and Semi-Quantitative Real-Time PCR

Corneal epithelium was removed with fine forceps following 30 minutes incubation with PBS containing 20 mM EDTA (Sigma-Aldrich) at 37° C. Next, 4-6 corneal stromas were pooled and lysed using BeadBug Microtube Homogenizer (Benchmark Scientific, Inc., Edison, N.J.). Next, RNA was isolated from the corneal stroma using RNeasy Plus Universal Mini kit (QIAGEN, Germantown, Md.). For isolating RNA from freshly sorted pDCs, purified cDCs, cultured pDCs, and cultured TGCs, RNeasy Plus Micro Kit (QIAGEN) was used. RNA yield was measured by spectroscopy (NanoDrop ND-1000; NanoDrop Technologies, Inc., Wilmington, Del.). cDNA was synthetized using 300 ng of template RNA using QuantiTect Reverse Transcription kit (Qiagen). qRT-PCR was performed using iTaq Universal SYBR Green Supermix (Biorad, Hercules, Calif.) and Eppendorf Mastercycler RealPlex 2 (Eppendorf, Hauppauge, N.Y.) with the primers described below. Relative mRNA levels were measured with ΔΔCT method.

### Single Cell PCR

8-10 corneas of naïve DPE-GFP×RAG1^{-/-} mice were excised and digested to yield a single cell suspension as described for flow cytometry experiments. GFP-tagged pDCs were sorted via Moflo Cell Sorter (Beckman Coulter) into a 96-well plate (100 cell/well) and cultured for 24 hours in RPMI (Gibco) supplemented with 10% 10% FBS (Gemini Bioproducts), 1% penicillin/streptomycin (Life Technologies), as well as 3 μg/ml Imiquimod, CpG-ODN, or control ODN (all InvivoGen). RNA isolation and cDNA synthesis were performed via REPLI-g Cell WGA & WTA kit (Qiagen). qRT-PCR was performed as described earlier. In another set of experiments, 4-6 corneas of DPE-GFP×RAG1^{-/-} mice undergoing inoculation of 10 μg Imiquimod, 10 μg CpG-ODN, or control ODN were harvested 24 hours following inoculation; next, corneas were digested and GFP-tagged pDCs were sorted (100 cell/condition) and underwent single cell PCR.

### pDC and TGCs Co-Culture Media ELISA

NGF levels in culture media of pDCs monoculture or TGCs and pDCs co-culture were measured via ChemiKine Nerve Growth Factor Sandwich ELISA (Millipore, Billerica, Mass.).

### Protein Extraction and Measurement of Phospho-CREB

On day 3 following culture of sorted pDCs, cells were lysed via Bio-Plex Pro Cell Signaling Reagent Kit (Biorad) supplemented with phosphotase inhibitor cocktail (Roche) and phospho-CREB and β-actin levels were measured via phospho-CREB and β-actin Bio-Plex assays (Biorad) according to the manufacturer's instructions. Relative phospho-CREB to total β-actin protein levels were normalized to control group.

### Statistical Analysis

Data was analyzed with SPSS version 17 (SPSS Inc., Chicago, Ill.). T test and ANOVA with Bonferroni host hoc were applied to assess differences among two or more groups, respectively, if assumptions were met. Chi square was used to compare categorical data. p less than 0.05 was considered significant.

### Subconjunctival Injections

Mice were anesthetized with intraperitoneal (i.p.) injection of 100 mg/kg Ketamine and 10-20 mg/kg Xylazine. After application of topical proparacaine hydrochloride, 30 ng DT (Sigma-Aldrich St. Louis, Mo.) in 10 μl PBS was administered subconjuctivally by means of a Nanofil syringe with 33-gauge needle to BDCA2-DTR mice to locally deplete pDCs. Injections were repeated every 48 hours to keep corneas pDC-depleted. WT C57BL/6 mice receiving DT and BDCA2-DTR mice receiving PBS served as control groups. Erythromycin ophthalmic ointment was applied on eye after injections. Mice were randomly assigned to study groups using a Random Number Table.

### Ocular Inoculation of TLR7 and 9 Agonists

Following anesthetizing DPE-GFP×RAG1^{-/-} mice and application of topical proparacaine hydrochloride, central corneal epithelium was debrided using an Algerbrush II corneal rust ring remover with a 0.5-mm burr (Alger Equipment Co, Lago Vista, Tex.). 10 μg Imiquimod (TLR7 agonist; InvivoGen, San Diego, Calif.), 10 μg phosphorothioate CpG 1826 oligonucleotide (CpG-ODN; a synthetic TLR9 agonist; InvivoGen), or control oligonucleotide 1826 (Control ODN; InvivoGen) was topically administered on the eye. 24 hours later corneas were removed to sort corneal GFP-tagged pDCs and single cell PCR experiments.

### Corneal Suture Placement

Under deep anesthesia and following application of topical proparacaine hydrochloride, corneal suture placement was performed on WT C57BL/6 mice as previously described (Cursiefen et al., Proc. Natl. Acad. Sci. U.S.A. 103(30):11405-10, 2006; Streilein et al., Invest. Ophthalmol. Vis. Sci. 37(2):413-424). Briefly, three 11-0 nylon sutures (Sharpoint; Vanguard, Houston, Tex.) were placed through the paracentral stroma of WT C57BL/6 mice, each 120° apart, without perforating the cornea, using aseptic microsurgical technique and an operating microscope.

### Corneal Thermal Cautery

As described previously (Streilein et al., Invest. Ophthalmol. Vis. Sci. 37(2):413-424; Williamson et al., Invest. Ophthalmic. Vis. Sci. 28(9):1527-1532, 1987), five light burns were induced on central 50% of the cornea of deeply anesthetized WT C57BL/6 mice after topical treatment with proparacaine hydrochloride, via the tip of a handheld thermal cautery (Aaron Medical Industries Inc., St. Petersburg, Fla.) under the dissecting microscope.

### Agarose Gel Electrophoresis

RNA extraction and cDNA synthesis were performed as described on GFP-tagged pDCs from the spleen naïve DPE-GFP×RAG1^{-/-} mice. PCR was performed in similar conditions described under qRT-PCR section using NGF primers. PCR products were run on 2% agarose gel. Gels were cast using 2% agarose (Sigma-Aldrich) in 0.5× Tris/borate/EDTA (TBE buffer) supplemented with 10 mM MgCl₂ and 0.5 mg/ml ethidium bromide (Sigma-Aldrich).

TABLE 1
Primers used in this study

| Transcript | Forward | Reverse |
|---|---|---|
| NGF | 5′-AGC ATT CCC TTG ACA CAG-3′ | 5′-GGT CTA CAG TGA TGT TGC-3′ |
| CREB | 5′-TGC AGC TGC CAC TCA GCC GGG-3′ | 5′-TGC CAA GCC AGT CCA TTC TCC AC-3′ |
| Sprr1a | 5′-GAA CCT GCT CTT CTC TGA GT-3′ | 5′-AGC TGA GGA GGT ACA GTG-3′ |
| Gap-43 | 5′-TGC TGT CAC TGA TGC TGC T-3′ | 5′-GGC TTC GTC TAC AGC GTC TT-3′ |
| Vimentin | 5′-TAC AGG AAG CTG CTG GAA GG-3′ | 5′-TGG GTG TCA ACC AGA GGA A-3′ |
| BDNF | 5′-CAA AGC CAC AAT GTT CCA CCA G-3′ | 5′-GAT GTC GTC GTC AGA CCT CTC G-3′ |
| GAPDH | 5′-CCC ACT AAC ATC AAA TGG GG-3′ | 5′-GAT GAT GAC CCT TTT GGC TC-3′ |

### Example 2: TLR7 and TLR9 Agonists—Angiogenesis

### Methods

### Splenic Cell Culture

Spleens of wildtype C57BL/6 mice were harvested and subjected to mechanical disruption. Samples were then filtered and underwent RBC lysis via incubation with ammonium chloride RBC lysis buffer (ACK; Biofluids, Rockville, Md.). Next, isolated splenic cells were cultured in RPMI supplemented with 10% heat-inactivated fetal bovine serum (FBS; Gemini Bioproducts, Woodland, Calif.), and 1% penicillin/streptomycin (Life Technologies, Carlsbad, Calif.). Cells were treated with 1 μg/ml Imiquimod (TLR7 agonist; InvivoGen, San Diego, Calif.), CpG-ODN (TLR9 agonist; InvivoGen), or PBS control for 24 hours. The experiment was done in triplicate.

### Flow Cytometry

After 24 hours of culture, isolated splenic cells were subjected to flow cytometry. In brief, cells were washed and then fixed and permeabilized using Foxp3/Transcription Factor Fixation/Permeabilization Reagent (eBioscience, San Diego, Calif.) according to the manufacturer's instructions. Cells were then washed twice with permeabilization buffer (eBioscience), blocked in 1% anti-CD16/CD32 FcR mAb at 4° C. in permeabilization buffer, and subsequently labeled with combinations of primary antibodies against CD45 (Biolegend, San Diego, Calif.), PDCA-1 (Biolegend), CD45R/B220 (Biolegend), Endostain (Abcam, Cambridge, Mass.) or their respective isotype controls for 30 minutes at 4° C. in the dark. After washing, cells were labeled with fluorochrome-conjugated anti-rabbit secondary antibody (for Endostatin) for 30 minutes at 4° C. All samples were washed and reconstituted in permeabilization before data acquisition with a BD LSR II flow cytometer (BD Biosciences). Data were analyzed with FlowJo V9.2 (FlowJo, LLC).

### Results

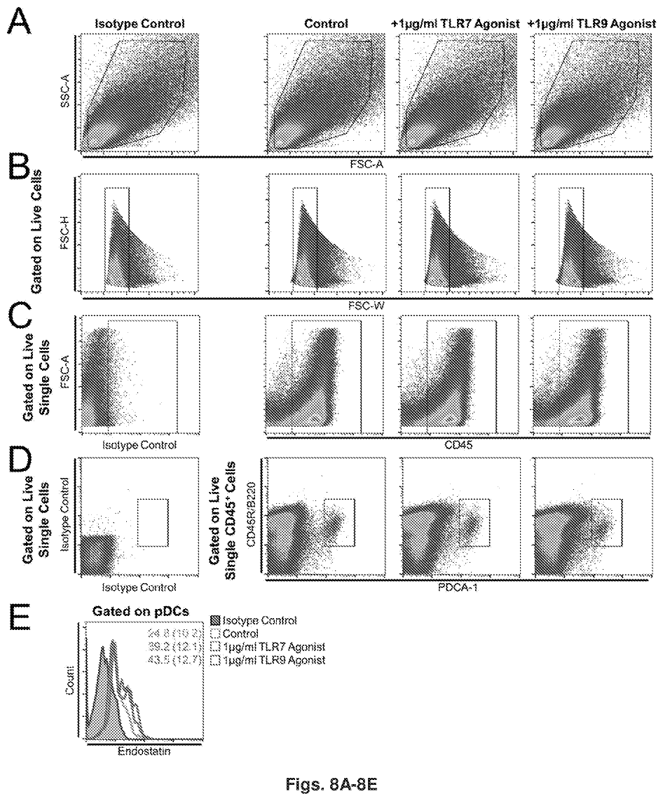

After gating out dead cells and debris (FIG. 8A), doublets were excluded (FIG. 8B). Next, CD45+ immune cells were selected (FIG. 8C). Plasmacytoid dendritic cells were subsequently identified by expression of PDCA-1⁺CD45R/B220⁺ (FIG. 8D). As demonstrated in FIG. 9, treatment of splenic cells with either Imiquimod (TLR7 agonist) or CpG-ODN (TLR9 agonist) increases the percentage of endostatin-secreting CD45⁺PDCA-1⁺CD45R/B220⁺ pDCs as well as the median fluorescent intensity of endostatin staining, showing expression of higher levels of endostatin by pDCs following treatment with TLR7 or TLR9 agonists.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

All publications and patent applications mentioned in this specification are herein incorporated by reference to the same extent as if each independent publication or patent application was specifically and individually indicated as being incorporated by reference in their entirety.

Use of singular forms herein, such as “a” and “the,” does not exclude indication of the corresponding plural form, unless the context indicates to the contrary. Similarly, use of plural terms does not exclude indication of a corresponding singular form.

Some embodiments are within the scope of the following numbered paragraphs.
1. A method of preventing or treating a disease or condition of the eye in a subject, the method comprising administering a toll-like receptor 7 (TLR7) agonist to an eye of the subject.
2. The method of paragraph 1, wherein the disease or condition of the eye is characterized by neovascularization.
3. The method of paragraph 2, wherein the neovascularization is corneal neovascularization.
4. The method of any one of paragraphs 1-3, wherein the subject has or is at risk of developing corneal infection, inflammation, autoimmune disease, limbal stem cell deficiency, neoplasm, dry eye disease, or ocular damage due to trauma, surgery, or contact lens wear.
5. The method of paragraph 2, wherein the neovascularization is retinal neovascularization.
6. The method of paragraph 2, wherein the neovascularization is choroidal neovascularization.
7. A method of preventing or treating a disease or condition of the eye in a subject, the method comprising administering a toll-like receptor 9 (TLR9) agonist to an eye of the subject, wherein the subject has or is at risk of developing corneal infection, inflammation, autoimmune disease, limbal stem cell deficiency, neoplasm, dry eye disease, or ocular damage due to trauma, surgery, or contact lens wear; or the disease or condition is characterized by retinal or choroidal neovascularization.
8. The method of any one of paragraphs 1, 2, 5, and 7, wherein the subject has or is at risk of developing ischemic retinopathy, diabetic retinopathy, retinopathy of prematurity, retinal vein occlusion, or macular degeneration.
9. The method of any one of paragraphs 1, 2, 6, and 7, wherein the subject has or is at risk of developing inflammatory neovascularization with uveitis.
10. The method of any one of paragraphs 1-9, wherein the disease or condition of the eye is characterized by ocular nerve degeneration or damage.
11. The method of paragraph 10, wherein the ocular nerve degeneration or damage is corneal nerve damage.
12. The method of paragraph 10 or 11, wherein the subject has or is at risk of developing dry eye disease, corneal infection, or corneal neurotrophic keratopathy.
13. The method of any one of paragraphs 10-12, wherein the subject has or is at risk of experiencing ocular damage due to trauma, surgery, or contact lens wear.
14. The method of any one of paragraphs 1-6 or 8-13, wherein the TLR7 agonist is selected from the group consisting of imiquimod, resiquimod, loxoribine, gardiquimod, and vesatolimod (GS-9620), GS-986, and combinations of two or more thereof.
15. The method of any one of paragraphs 7-13, wherein the TLR9 agonist is a CpG oligonucleotide or a combination thereof.
16. The method of paragraph 15, wherein the CpG oligonucleotide is selected from the group consisting of: CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.
17. The method of any one of paragraphs 1-16, wherein the method further comprises administering a plasmacytoid dendritic cell (pDC) to an eye of the subject.
18. The method of paragraph 17, wherein the plasmacytoid dendritic cell is applied to the cornea of the subject.
19. The method of paragraph 18, wherein the plasmacytoid dendritic cell is administered to the subject by intravitreal or sub-retinal injection.
20. The method of any one of paragraphs 1-19, wherein the subject is a human subject.
21. The method of any one of paragraphs 17-20, wherein the plasmacytoid dendritic cell is obtained from the subject to whom it is administered.
22. The method of any one of paragraphs 17-20, wherein the plasmacytoid dendritic cell is obtained from an individual and/or species different from the subject to whom it is administered.
23. The method of any one of paragraphs 1-22, comprising administration of a TLR7 agonist in combination with a TLR9 agonist.
24. A composition comprising a TLR7 agonist and/or a TLR9 agonist and an ophthalmic carrier or diluent.
25. The composition of paragraph 24, wherein the carrier or diluent is selected from the group consisting of glycerin, hydroxyethylcellulose (HEC), hydroxypropyl methylcellulose (HPMC), polyvinyl alcohol (PVA), carboxy methylcellulose (CMC), sodium chloride, polyvidone, polyethylene glycol, propylene glycol, hypromelloses, boric acid, sodium borate, sodium hyaluronate, Hamamelis virginiana, and tissue glue.
26. The composition of paragraph 24 or 25, further comprising a preservative selected from the group consisting of benzalkonium (BAK), poloxamer 407, potassium sorbate, polyquad, sodium perborate, purite, cetrimide, hydroxypropyl guar, and polyquaternium
27. A kit comprising the composition of any one of paragraphs 24-26 and a topical anesthetic eye drop.
28. A kit comprising the composition of any one of paragraphs 24-27 and a syringe, dropper, or applicator for administration of the composition.

Other embodiments are within the scope of the following claims.

1. A method of preventing or treating a disease or condition of the eye in a subject, the method comprising administering a toll-like receptor 7 (TLR7) agonist to an eye of the subject.

2. The method of claim 1, wherein the disease or condition of the eye is characterized by neovascularization.

3. The method of claim 2, wherein the neovascularization is corneal neovascularization.

4. The method of claim 1, wherein the subject has or is at risk of developing corneal infection, inflammation, autoimmune disease, limbal stem cell deficiency, neoplasm, dry eye disease, or ocular damage due to trauma, surgery, or contact lens wear.

5. The method of claim 2, wherein the neovascularization is retinal neovascularization.

6. The method of claim 2, wherein the neovascularization is choroidal neovascularization.

7. A method of preventing or treating a disease or condition of the eye in a subject, the method comprising administering a toll-like receptor 9 (TLR9) agonist to an eye of the subject, wherein the subject has or is at risk of developing corneal infection, inflammation, autoimmune disease, limbal stem cell deficiency, neoplasm, dry eye disease, or ocular damage due to trauma, surgery, or contact lens wear; or the disease or condition is characterized by retinal or choroidal neovascularization.

8. The method of claim 1, wherein the subject has or is at risk of developing ischemic retinopathy, diabetic retinopathy, retinopathy of prematurity, retinal vein occlusion, or macular degeneration.

9. The method of claim 1, wherein the subject has or is at risk of developing inflammatory neovascularization with uveitis.

10. The method of claim 1, wherein the disease or condition of the eye is characterized by ocular nerve degeneration or damage, wherein optionally: (a) the disease or condition is corneal nerve damage, (b) the subject has or is at risk of developing dry eye disease, corneal infection, or corneal neurotrophic keratopathy, or (c) the subject has or is at risk of experiencing ocular damage due to trauma, surgery, or contact lens wear.

11. (canceled)

12. (canceled)

13. (canceled)

14. The method of claim 1, wherein the TLR7 agonist is selected from the group consisting of imiquimod, resiquimod, loxoribine, gardiquimod, and vesatolimod (GS-9620), GS-986, and combinations of two or more thereof.

15. The method of claim 7, wherein the TLR9 agonist is a CpG oligonucleotide or a combination thereof, wherein optionally the CpG oligonucleotide is selected from the group consisting of: CpG-ODN 2216, CpG-ODN 2336, CpG-ODN 2006 (CpG ODN 7909=PF-3512676), CpG-ODN D-SL01, CpG-ODN 2395, CpG-ODN M326, CpG-ODN D-SL03, ISS 1018 CpG ODN, IMO-2055, CpG-28, CPG10101, IMO-2125, SD-101, CpG 7909, and CYT003-QbG10.

16. (canceled)

17. The method of claim 1, wherein the method further comprises administering a plasmacytoid dendritic cell (pDC) to an eye of the subject.

18. The method of claim 17, wherein the plasmacytoid dendritic cell is applied to the cornea of the subject or is administered to the subject by intravitreal or sub-retinal injection.

19. (canceled)

20. The method of claim 1, wherein the subject is a human subject.

21. The method of claim 17, wherein the plasmacytoid dendritic cell is obtained from the subject to whom it is administered.

22. The method of claim 17, wherein the plasmacytoid dendritic cell is obtained from an individual and/or species different from the subject to whom it is administered.

23. The method of claim 1, comprising administration of a TLR7 agonist in combination with a TLR9 agonist.

24. A composition comprising a TLR7 agonist and/or a TLR9 agonist and an ophthalmic carrier or diluent, wherein optionally the carrier or diluent is selected from the group consisting of glycerin, hydroxyethylcellulose (HEC), hydroxypropyl methylcellulose (HPMC), polyvinyl alcohol (PVA), carboxy methylcellulose (CMC), sodium chloride, polyvidone, polyethylene glycol, propylene glycol, hypromelloses, boric acid, sodium borate, sodium hyaluronate, Hamamelis virginiana, and tissue glue, and wherein optionally, the composition further comprises a preservative selected from the group consisting of benzalkonium (BAK), poloxamer 407, potassium sorbate, polyquad, sodium perborate, purite, cetrimide, hydroxypropyl guar, and polyquaternium.

25. (canceled)

26. (canceled)

27. A kit comprising the composition of claim 24 and a topical anesthetic eye drop, wherein the kit optionally comprises a syringe, dropper, or applicator for administration of the composition.

28. (canceled)
